(19) United States
(12) Patent Application Publication
(10) Pub. No.: US 2020/0362309 A1
(43) Pub. Date: Nov. 19, 2020

(54) Method for preparing BAP or BA cells

(71) Applicant: Anagenesis Biotechnologies S.A.S., Illkirch-Graffenstaden (FR)

(72) Inventors: Anne-Laure Fabienne Bernadette Hafner, Illkirch-Graffenstaden (FR); Lionel Adolphe Théodore Meyer, Illkirch-Graffenstaden (FR); Aurore Sabine Hick, Illkirch-Graffenstaden (FR)

(73) Assignee: Anagenesis Biotechnologies S.A.S., Illkirch-Graffenstaden (FR)

(21) Appl. No.: 16/958,160

(22) PCT Filed: Dec. 24, 2018
(86) PCT No.: PCT/EP2018/086854

§ 371 Date: Jun. 26, 2020

(30) Foreign Application Priority Data
Dec. 29, 2017 (EP) 17211081.9

(51) Int. Cl. C12N 5/077; A61K 35/35

(52) U.S. Cl. CPC C12N 5/0653; A61K 35/35; C12N 2506/45; C12N 2500/38; C12N 2501/39; C12N 2501/999; C12N 2501/115; C12N 2501/12; C12N 2501/105; C12N 2500/25; C12N 2501/11

(57) ABSTRACT

The invention relates to a method for preparing BAP or BA cells, obtained BAP or BA cell populations and their use as a medicament.

### FIELD OF THE INVENTION

The present invention relates to a method for preparing BAP (Brown Adipocyte Progenitor) or BA (Brown Adipocyte) cells, BAP or BA cell populations and their use as a medicament.

### BACKGROUND OF THE INVENTION

In mammals, two main types of adipocytes coexist, i.e. brown adipocytes (BA) and white adipocytes (VVA), which are all involved in energy balance regulation while having opposite functions. White adipose tissue (WAD is dispersed throughout the body and is mainly involved in energy storage. In contrast to WAT, brown adipose tissue (BAT) is specialized in energy expenditure. Activated BAT consumes metabolic substrate and burns fat to produce heat via the uncoupling protein (UCP)-1. This tissue is found in large quantities in newborns and hibernating species. In humans, the quantity of BAT decreases over time, and only small deposits can be found in adults.

BA have a significant therapeutic potential; in burning fat to generate heat and regulating the body's homeothermy, they have been shown to promote weight loss or regulate metabolic parameters such as glycemia. Therefore, a cellular source of BAPs and/or BAs is urgently needed for the clinic.

### SUMMARY OF THE INVENTION

The present invention relates to a method for preparing BAP cells, said method comprising the following steps:
a) Culturing pluripotent cells in a culture medium comprising an activator of the Wnt signaling pathway to obtain induced paraxial mesoderm progenitor (iPAM) cells,
b) Culturing said iPAM cells in a myogenic culture medium,
c) Optionally further culturing cells obtained at the end of step b) in a culture medium comprising serum or an equivalent thereof, optionally further comprising FGF2 or an equivalent thereof,
d) Selecting BAP cells by passaging the cells obtained at the end of step b) or c) and seeding them into culture dish.

The present invention also relates to a method for preparing BA cells, said method preferably comprising steps a), b), optionally step c), step d) as defined in the method for obtaining BAP cells and subsequently comprising the following step:
e) Culturing the selected BAP cells preferably those obtainable at the end of step d) in an adipogenic culture medium comprising serum or an equivalent thereof obtaining BA cells.

In an embodiment, step a) may be carried out in a culture medium further comprising an inhibitor of the Bone Morphogenetic Pathway (BMP) signaling pathway and optionally DMSO.

In a preferred embodiment:
a) the Wnt signaling pathway is the canonical Wnt/beta catenin signaling pathway and/or the Wnt/PCP signaling pathway,
b) the inhibitor or the BMP signaling pathway is selected from the group consisting of: Noggin, Chordin, Chordin-like 1-3, Follistatin, Follistatin-like 1-5, a member of the Dan family and variants and fragments thereof.

In a further embodiment, the myogenic culture medium used in step b) comprises or consists of or essentially consists of a culture medium, serum or an equivalent thereof, an inhibitor of a BMP receptor, an activator of the c-MET receptor and an activator of an IGF or insulin receptor.

In an embodiment, a method is carried out wherein the adipogenic culture medium of step e) comprises or essentially consists of a culture medium, an inhibitor of the TGFbeta/Activin/NODAL pathway (preferably SB431542), an activator of the EGF receptor (preferably EGF (Epidermal Growth Factor)), ascorbic acid, and an activator of a corticoid receptor (preferably hydrocortisone).

In a further embodiment, BA cells or the population of BA cells are characterized by the expression of UCP1.

The invention also relates to a population of BA cells obtainable by the method as defined herein, said population of cells comprising at least 10%, 15%, 20%, 25%, 30%, 35%, 40%, 45%, 50%, 55%, 60% 65%, 70%, 75%, 80%, 85%, 90%, 95%, 99% or 100% of cells expressing UCP1.

The invention also relates to a population of BAP cells obtainable by the method as defined herein characterized by the ability of said population to be converted into a BA population as defined herein.

Preferably, said population of BAP or BA cells is for use as a medicament. More preferably, the medicament is for treating a disease or condition linked with BA or BAP cell activity and preferably being a metabolic disease or condition such as obesity-related pathologies, metabolic syndrome, diabetes mellitus, hyperlipidemia, NASH (Non-Alcoholic Steato Hepatitis), Energy balance (intake versus expenditure).

The invention also relates to the use of the population of BAP or BA cells as defined herein for screening purposes.

### DETAILED DESCRIPTION OF THE INVENTION

### Method for Preparing BAP Cells

In a first aspect there is provided a method for preparing BAP cells, said method comprising the following steps:
a) Culturing pluripotent cells in a culture medium comprising an activator of the Wnt signaling pathway to obtain induced paraxial mesoderm progenitor (iPAM) cells,
b) Culturing said iPAM cells in a myogenic culture medium,
c) Optionally further culturing cells obtained at the end of step b) in a culture medium comprising serum or an equivalent thereof, further optionally comprising FGF2 or an equivalent thereof,
d) Selecting BAP cells by passaging the cells obtained at the end of step b) or c) and seeding them into culture dish.

In a second aspect, there is provided a method for preparing BA cells, wherein in a preferred embodiment BAP cells are prepared using the method defined above (i.e. step a), step b), optional step c) and step d)) said method further comprising the following step:
e) Culturing the selected BAP cells obtainable at the end of step d) in an adipogenic culture medium comprising serum or an equivalent thereof obtaining BA cells.

Within the context of both methods, one refers to the preparation of BAP or BA cells. The expression BAP or BA cells may be replaced by a population comprising or consisting of or essentially consisting of BAP or BA cells. BAP and BA cells are later identified herein.

Unless otherwise indicated when one refers to a or to the method of the invention, one refers to a or the method for of preparing BAP or for preparing BA cells unless otherwise indicated.

Step a

Cells cultured in step a) of a method of the invention are preferably pluripotent cells. The term “pluripotent cells” as used herein refers to mammalian undifferentiated cells which can give rise to a variety of different cell lineages. Typically, pluripotent cells may express the following markers Oct4, SOX2, Nanog, SSEA 3 and 4, TRA 1/81, see International Stem Cell Initiative recommendations, 2007. The expression or the presence of a given marker in a cell may be assessed as disclosed in the general part entitled “definitions to be applied in the context of the application”.

In one embodiment, the pluripotent cells are mammalian pluripotent cells. Preferably said pluripotent cells are human pluripotent cells. In another embodiment, the pluripotent cells are non-human mammalian pluripotent cells.

In one embodiment, the pluripotent cells are stem cells. Preferably, said stem cells are embryonic stem cells. Alternatively, said stem cells are adult stem cells. In the case adult stem cells are used, it means that BAP or BA cells may be generated from organ restricted stem cells or mesenchymal stem cells (MSCs).

In another embodiment, the pluripotent cells are human embryonic stem cells (hES cells). In another embodiment, the pluripotent cells are non-human mammalian embryonic stem cells. Typically, hES cell lines (Loser et al., 2010) such as the one described in the following table may be employed for the method of the invention:

| | | passage | country of | |
|---|---|---|---|---|
| line | karyotype | available | origin | origin |
| SA01 | 46XY | 25 | Sweden | Cellartis AB |
| VUB01 | 46XY | 73 | Belgium | AZ-VUB Bruxel |
| HUES 24, | 46XY | 26 | USA | Harvard |
| H1 | 46XY, | 26 | USA | Wicell research |
| | 20q11.21 | | | Institute |
| H9 | 46XX | 27 | USA | Wicell research |
| | | | | Institute |
| WT3 | 46XY | 35 | UK | UKSCB |
| HUES1 | 46XX | 33 | USA | Harvard |

In one embodiment, the pluripotent cells are non-human embryonic stem cells, such as mouse stem cells, rodent stem cells or primate stem cells.

In one embodiment, the pluripotent cells are induced pluripotent stem cells (iPSC). Induced pluripotent stem cells (iPSC) are a type of pluripotent stem cells artificially derived from a non-pluripotent, typically an adult somatic cell, by inducing a “forced” expression of certain genes. iPSC were first produced in 2006 from mouse cells (Takahashi and Yamanaka, 2006) and in 2007 from human cells (Takahashi et al., 2007; Yu et al., 2007).

As used herein, the term “Wnt signaling pathway” denotes a signaling pathway which may be divided in two pathways: the “canonical Wnt/beta catenin signaling pathway” and the “Wnt/PCP signaling pathway”. As used herein, the term “canonical Wnt/beta catenin signaling pathway” or “Wnt/PCP signaling pathway” in its general meaning denotes a network of proteins and other bioactive molecules (lipids, ions, sugars . . . ) best known for their roles in embryogenesis and cancer, but also involved in normal physiological processes in adult animals. The “canonical Wnt/beta catenin signaling pathway” is characterized by a Wnt dependant inhibition of glycogen synthase kinase 3β (GSK-3β), leading to a subsequent stabilization of β-catenin, which then translocates to the nucleus to act as a transcription factor. The “Wnt/PCP signaling pathway” does not involve GSK-3β or β-catenin, and comprises several signaling branches including Calcium dependant signaling, Planar Cell Polarity (PCP) molecules, small GTPases and C-Jun N-terminal kinases (JNK) signaling. These pathways are well described in numerous reviews such as (Clevers, 2006; Montcouquiol et al., 2006; Schlessinger et al., 2009).

In one embodiment, the Wnt signaling pathway is the canonical Wnt/β-catenin signaling pathway.

In another preferred embodiment, the Wnt signaling pathway is the Wnt/PCP signaling pathway. In another preferred embodiment, the Wnt signaling pathway is the canonical Wnt/β-catenin signaling pathway and Wnt/PCP signaling pathway.

As used herein the term “activator” or “activator of the Wnt signaling pathway” (unless otherwise indicated) denotes a substance that enhances or promotes or activates a Wnt signaling activity. For example, for the canonical Wnt/β-catenin signaling pathway, this activity can be measured by Wnt reporter activity using established multimers of LEF/TCF binding sites reporters, and/or inhibition of GSK-3β, and/or activation of canonical Wnt target genes such as T, Tbx6, Msgn1, or Axin2. An activation of a Wnt signaling activity may therefore be assessed as being an increase of a Wnt of Msgn1 reporter activity (Chal J et al 2015) as identified above. Said increase may be of at least 1%, 5% 10%, 15%, 20%, 25%, 30%, 35%, 40%, 45%, 50%, 55%, 60%, 65%, 70%, 75%, 80%, 85%, 90%, 95%, 100% or more.

In an embodiment, the activator of the canonical Wnt/β-catenin signaling pathway or the Wnt/PCP signaling pathway according to the invention is a member of the R-spondin family, originating from a vertebrate species or modified.

In an embodiment, the member of the R-spondin family is a member of the mammalian R-spondin family. In a particular embodiment, the member of the R-spondin family according to the invention is selected in the group consisting of R-spondin 1, R-spondin 2, R-spondin 3 and R-spondin 4. In a particular embodiment, the member of the R-spondin family according to the invention is R-spondin 3. In a particular embodiment, the member of the R-spondin family according to the invention is R-spondin 2.

Vertebrate recombinant R-spondins can be purchased commercially, or produced as conditioned culture medium. This involves expressing a construct containing the coding sequence of an R-spondin protein into competent cells, such as COS cells. R-spondin protein is secreted in the culture medium. Conditioned medium can be applied directly to pluripotent cells or prediluted in basal medium.

As used herein, the term “R-spondin3” or “R-spondin2” refers to members of the family of secreted proteins in vertebrates that activate the Wnt signaling pathway. An exemplary sequence for human R-spondin3 protein is deposited in the database under accession number NP_116173.2 (SEQ ID NO:1). An exemplary sequence for mouse R-spondin3 protein is deposited in the database under accession number NP_082627.3 (SEQ ID NO:2). An exemplary sequence for human R-spondin2 protein is deposited in the database under accession number NP_848660.3 (SEQ ID NO:3). An exemplary sequence for mouse R-spondin2 protein is deposited in the database under accession number NP_766403.1 (SEQ ID NO:4).

As used herein, the term “R-spondin3” also encompasses any functional variants of R-spondin3 wild type (naturally occurring) protein, provided that such functional variants retain the advantageous properties of differentiating factor for the purpose of the present invention. In one embodiment, said functional variants are functional homologues of R-spondin3 having at least 60%, 70%, 80%, 90%, 91%, 92%, 93%, 94%, 95%, 96%, 97%, 98%, 99% or 100% identity to the most closely related known natural R-spondin3 polypeptide sequence, for example, to human or mouse polypeptide R-spondin3 of SEQ ID NO:1 or SEQ ID NO:2 respectively, and retaining substantially the same Wnt activation activity as the related wild type protein. In another embodiment, said functional variants are fragments of R-spondin3, for example, comprising at least 50, 100, or 200 consecutive amino acids of a wild type R-spondin3 protein, and retaining substantially the same Wnt activation activity. In another embodiment, such functional variant can consist in R-spondin3 gene product isoforms such as the isoform 2 of the human R-spondin3 as described under the ref. Q9BXY4-2 and CAI20142.1 (SEQ ID NO:5). In this context “substantially” preferably means that an activity of such a functional variant is at least 40%, 50%, 60%, 70%, 80%, 90% or 100% of the activity of the wild type or naturally occurring molecule it derives from. In this context, an activity of the wild type or naturally occurring molecule it derives from preferably refers to the activity to activate the Wnt signaling pathway.

As used herein, the term “R-spondin2” also encompasses any functional variants of R-spondin2 wild type (naturally occurring) protein, provided that such functional variants retain the advantageous properties of differentiating factor for the purpose of the present invention. In one embodiment, said functional variants are functional homologues of R-spondin2 having at least 60%, 70%, 80%, 90%, 91%, 92%, 93%, 94%, 95%, 96%, 97%, 98%, 99% or 100% identity to the most closely related known natural R-spondin2 polypeptide sequence, for example, to human or mouse polypeptide R-spondin2 of SEQ ID NO:3 or SEQ ID NO:4 respectively, and retaining substantially the same Wnt activation activity as the related wild type protein. In another embodiment, said functional variants are fragments of R-spondin2, for example, comprising at least 50, 100, or 200 consecutive amino acids of a wild type R-spondin2 protein, and retaining substantially the same Wnt activation activity. In another embodiment, said functional variants can consist in R-spondin2 gene product isoforms such as the isoform 2 or the isoform 3 of the human R-spondin2 such as described respectively under the ref. Q6UXX9-2 (SEQ ID NO:6) or under the ref. Q6UXX9-3 (SEQ ID NO:7).

In an embodiment, the activator used in step a) of a method of the invention is a combination of the R-spondin 3 and R-spondin 2. In an embodiment, the activator used in step a) of a method of the invention may be the human R-spondin-3 isoform 2 of sequence SEQ ID NO:5. In an embodiment, the activator used in step a) of a method of the invention may be the human R-spondin-2 isoform 2 of sequence SEQ ID NO:6, or the human R-spondin-2 isoform 3 of sequence SEQ ID NO:7.

In another embodiment, introducing directly into the cells environment an appropriate amount of pharmacological GSK-3β inhibitor, for example the chemical compound CHIR99021 or an equivalent thereof is used as an alternative for increasing the activity of Wnt signaling pathway in the system, alone or in combination with R-spondin. An equivalent of CHIR99201 is CHIR98014 described in Huang et al 2017.

As used herein, the term “GSK-3 β” for “Glycogen synthase kinase 3 beta” denotes a serine/threonine protein kinase that mediates the addition of phosphate molecules on certain serine and threonine amino acids on particular cellular substrates. It is well known in the art that an inhibitor of GSK-3β may activate the Wnt signaling pathway, see for example (Cohen and Goedert, 2004; Sato et al., 2004; Taelman et al., 2010; Wu and Pan, 2010).

In a preferred embodiment, the inhibitor of GSK-3β is CHIR99021 or an equivalent thereof.

As used herein the term “induced Paraxial Mesoderm progenitor cells” or “iPAM” refers to cells derived from any cell type but exhibiting characteristics of progenitor cells of the Paraxial Mesoderm. In one embodiment, the iPAM cells are characterized by the following properties:
a) they express biomarkers characteristic of Paraxial mesoderm progenitor cells such as Tbx6, EphrinA1, EphrinB2, EPHA4, PDGFRalpha, Sall1, Sall4, Dll1, Dll3, Papc (Pcdh8), Lfng, Hes7, Ripply1, Ripply2, Brachyury (T), Cdx2, Cdx4, Evx1, Cxcr4, III7rd, Fgf8, Fgf17, Gbx2, Wnt3a, Wnt5b, Rspo3, SP5, SP8, Has2, Dkk1, Dact1, Pax3, Pax7, Mesp1, Mesp2 or Msgn1 genes. Preferentially Msgn1 gene as measured for example with a gene reporter assay comprising the Msgn1 promoter (Chat J et al 2015), and;
b) they are multipotent cells, capable of differentiating into at least skeletal, dermis or muscle cell lineages;
c) optionally, they may have long term self-renewal properties, e.g., they can be maintained in culture more than 6 months.

The multipotency of said induced Paraxial Mesoderm progenitor (iPAM) cells can be tested in vitro, e.g., by in vitro differentiation into skeletal, dermal or muscle cell lineages using the protocols defined for example in WO 2013/030243. In the current invention, we demonstrated that these iPAM cells can differentiate into BAP and BA cells.

As used herein, the term “multipotent” refers to cells that can differentiate in more than one cell lineage depending on the environmental and culture conditions. Contrary to induced and embryonic stem cells which are pluripotent and can differentiate into all types of somatic cell lineages, the induced paraxial mesoderm progenitor cells of the present invention have limited differentiation capacity.

In an embodiment, the concentration of R-spondin3 used for culture of pluripotent cells in step a) is from 0.1 ng/ml and 500 ng/ml, preferably from 1 ng/ml and 500 ng/ml and more preferably from 5 ng/ml and 30 ng/ml.

In an embodiment, the concentration of R-spondin2 used for culture of pluripotent cells in step a) is from 1 ng/ml and 500 ng/ml, preferably from 5 ng/ml and 30 ng/ml. In an embodiment, the concentration of R-spondin3 or R-spondin2 is about 10 ng/ml or is 10 ng/ml. With a concentration of 10 ng/ml, more than 50% up to 70% of pluripotent cells are differentiated in induced Paraxial Mesoderm progenitor (iPAM) cells.

In an embodiment, pluripotent cells are cultured with R-spondin3 or R-spondin2 during 1 to 15 days, or for a shorter time period. In a particular embodiment, pluripotent cells are cultured with R-spondin3 or/and R-spondin2 during at least 10 days at a concentration of 10 ng/ml.

In an embodiment, the concentration of CHIR99021 is from 1 to 5 μM, or from 2 to 4 μM or 3 μM.

In a preferred embodiment, the culture medium of step a) further comprises an inhibitor of the Bone Morphogenetic Protein (BMP) signaling pathway and optionally DMSO.

As used herein, the term “inhibitor of the BMP signaling pathway” (also called “an inhibitor” unless otherwise indicated) denotes any compound, natural or synthetic, which results in a decreased activation of the BMP (bone morphogenetic protein) signaling pathway, which is characterized by the binding of a dimer BMP protein to a heterocomplex constituted of BMP type I and type II receptors, which results in a phosphorylation cascade leading to the phosphorylation of Smad1/5/8, and resulting in target genes activation, such as Id genes. Typically, an inhibitor of the BMP signaling pathway provokes a decrease in the levels of phosphorylation of the proteins Smad 1, 5 and 8 (Gazzero and Minetti, 2007).

The skilled person in the art knows how to assess whether a given compound is an inhibitor of the BMP signaling pathway. Typically, a compound is deemed to be an inhibitor of the BMP signaling pathway if, after culturing cells in the presence of said compound, the level of phosphorylated Smad 1, 5 or 8 is decreased compared to cells cultured in the absence of said compound. Levels of phosphorylated Smad proteins can be measured by Western blot using antibodies specific for the phosphorylated form of said Smad proteins. The decrease may be of at least 5%, 10%, 15%, 20%, 25%, 30%, 35%, 40%, 45%, 50%, 55%, 60%, 65%, 70%, 75%, 80%, 85%, 90%, 95% or 100% or more.

Target genes activation, such as Id genes, can typically be measured by direct Id1/2/3 transcripts (mRNA) production, via quantitative real-time PCR (qRT-PCR) and expression levels can be compared to control situation, in the absence of said compound.

The inhibitor of the BMP signaling pathway may be a BMP antagonist, a chemical compound that blocks BMP type I and/or type II receptors activity (BMP type I/II receptor inhibitor), an inhibitor of BMP type I and/or type II gene expression, or a molecule which inhibits any downstream step of the BMP signaling pathway. The inhibitor of BMP signaling may be a natural or a synthetic compound. When the inhibitor of the BMP signaling pathway is a protein, it may be a purified protein or a recombinant protein or a synthetic protein.

In one embodiment, the inhibitor of the BMP signaling pathway is a BMP type I receptors inhibitor.

Many methods for producing recombinant proteins are known in the art. The skilled person can readily, from the knowledge of a given protein's sequence or of the nucleotide sequence encoding said protein, produce said protein using standard molecular biology and biochemistry techniques.

In one embodiment of the invention, the inhibitor of the BMP signaling pathway is selected from the group consisting of Noggin, Chordin and related proteins (Chordin-like 1/2/3), Follistatin and related proteins (Follistatin-like 1/2/3/4/5), proteins of the Dan family (including Cerberus1, Gremlin 1 and 2, Cerl-2 (Coco), SOST (Sclerostin), SOSTDC1 (Wise)) and variants and fragments thereof which inhibit the BMP signaling pathway.

In another embodiment of the invention, the inhibitor of the BMP signaling pathway is selected from the group consisting of BMP-1/Tolloid-like proteins, TWSG1 (twisted gastrulation), TMEFFs (Tomoregulins), Biglycan, TSK (Tsukushi), BMPER (Crossveinless 2), Ogon (Sizzled), AMN (Amnionless), CTGF (Connective Tissue Growth Factor), and HSPGs (including Glypican3 and Syndecan4).

In another embodiment, the inhibitor of the BMP signaling pathway is noggin. Noggin can be a mammalian noggin, preferably murine noggin (mouse noggin exemplified by GenPept accession number NP_032737, SEQ ID NO:10) or human noggin (human noggin exemplified by GenPept accession number EAW94528, SEQ ID NO:11). It may be purified or recombinant. It may be in monomeric or dimeric form.

In one embodiment, the inhibitor of the BMP signaling pathway is a compound that inhibits BMP signaling transduction cascade. In an embodiment, the compound that inhibits BMP signaling transduction cascade is a synthetic or a chemical compound.

In another embodiment, the inhibitor of the BMP signaling pathway is an inhibitor of BMP type I receptors. As used herein, the term “BMP type I receptors” for “Bone Morphogenetic Protein” denotes transmembrane proteins with serine/threonine protein kinase activity that mediates the addition of phosphate molecules on certain serine and threonine amino acids on particular cellular substrates. It is well known in the art that an inhibitor of BMP type I receptors may block the BMP signaling pathway, see for example Yu BP et al, 2008. In a preferred embodiment, the inhibitor of BMP type I receptors is Dorsomorphin, a chemical compound or any derivatives generated by structure-activity studies [Cuny G D et al., 2008]. Dorsomorphin (6-[4-(2-Piperidin-1-yl-ethoxy)phenyl]-3-pyridin-4-yl-pyrazolo[1,5-a]pyrimidine, also known as Compound C) is inhibiting specifically BMP type I receptors (ALK2, 3, and 6) [Yu PB et al., 2008]. A preferred inhibitor or a BMP receptor is LDN193189. LDN193189 is an inhibitor of the BMP type I receptor Alk2 and Alk3. Recombinant Noggin can be purchased from R&D Systems or Peprotech or can be produced using standard techniques as described above.

Typically, the inhibitor of the BMP signaling pathway is added to the culture medium of step a) of a method of the invention in a concentration ranging from 1 to 10000 ng/ml, preferably from 5 to 1000 ng/ml, preferably from 5 to 500 ng/ml, preferably from 10 to 200 ng/ml, even more preferably at about 200 ng/ml.

Typically, noggin is added to the culture medium of step a) of a method of the invention at a concentration ranging from 1 to 1000 ng/ml, preferably from 10 to 200 ng/ml, even more preferably at about 200 ng/ml or at 200 ng/ml.

Typically, Dorsomorphin is added to the culture medium of step a) of a method of the invention in a concentration ranging from 0.1 to 2 μM, preferably at 1 μM. Concentrations of LDN193189 may be from 300 to 600 nM or from 400 to 500 nM or about 500 nM, or 500 nM.

In one embodiment, pluripotent cells are cultured with the inhibitor of the BMP signaling pathway during 1 to 4 days.

In an embodiment, the culture medium of step a) comprises a Wnt activator and an inhibitor of BMP signaling pathway according to the invention to improve the differentiation of pluripotent cells into induced Paraxial Mesoderm progenitor (iPAM) cells. In an embodiment, the method is such that:
a) the Wnt signaling pathway is the canonical Wnt/beta catenin signaling pathway and/or the Wnt/PCP signaling pathway,
b) the inhibitor or the BMP signaling pathway is selected from the group consisting of:

Noggin, Chordin, Chordin-like 1-3, Follistatin, Follistatin-like 1-5, a member of the Dan family and variants and fragments thereof.

In an embodiment, the Wnt activator is R-spondin3 and the inhibitor of BMP signaling pathway is Noggin.

In another embodiment, the culture medium used in step a) may further comprise DMSO (Dimethyl sulfoxide) or an equivalent of the DMSO to further improve the differentiation of pluripotent cells into induced Paraxial Mesoderm progenitor (iPAM) cells. As used herein, the term “equivalent” means a substance exhibiting the same properties as DMSO which is a solvent that dissolves both polar and nonpolar compounds.

In an embodiment, the culture medium used in step a) comprises R-spondin 3, Noggin and DMSO to improve the differentiation of pluripotent cells into induced Paraxial Mesoderm progenitor (iPAM) cells.

In another embodiment, the culture medium used in step a) comprises R-spondin 3 and DMSO to improve the differentiation of pluripotent cells into induced Paraxial Mesoderm progenitor (iPAM) cells.

In another embodiment, the culture medium used in step a) comprises R-spondin 2, Noggin and DMSO to improve the differentiation of pluripotent cells into induced Paraxial Mesoderm progenitor (iPAM) cells.

In another embodiment, the culture medium used in step a) comprises R-spondin 2 and DMSO to improve the differentiation of pluripotent cells into induced Paraxial Mesoderm progenitor (iPAM) cells.

In another embodiment, the culture medium used in step a) comprises R-spondin 3, Dorsomorphin and DMSO to improve the differentiation of pluripotent cells into induced Paraxial Mesoderm progenitor (iPAM) cells.

In another embodiment, the culture medium used in step a) comprises R-spondin 2, Dorsomorphin and DMSO to improve the differentiation of pluripotent cells into induced Paraxial Mesoderm progenitor (iPAM) cells.

In still another embodiment, the culture medium used in step a) comprises R-spondin 3, R-spondin 2, Noggin and DMSO to improve the differentiation of pluripotent cells into induced Paraxial Mesoderm progenitor (iPAM) cells.

In still another embodiment, the culture medium used in step a) comprises R-spondin 3, R-spondin 2 and DMSO to improve the differentiation of pluripotent cells into induced Paraxial Mesoderm progenitor (iPAM) cells.

In still another embodiment, the culture medium used in step a) comprises R-spondin 3, R-spondin 2, Dorsomorphin and DMSO to improve the differentiation of pluripotent cells into induced Paraxial Mesoderm progenitor (iPAM) cells.

In another preferred embodiment, the following alternatives may be used for increasing the activity of R-spondin factor in the system:
1. enhancing endogenous expression of the gene encoding said R-spondin factor or a modified form of R-spondin,
2. allowing ectopic expression of said R-spondin factor by introducing an expression vector comprising a coding sequence of R-spondin factor operably linked to control sequences into the pluripotent cells to be differentiated, or by introducing in the cells coding RNA for R-spondin factor
3. introducing directly into the cells environment an appropriate amount of R-spondin factor, for example as recombinant R-spondin factor (family of R-spondinl, 2 ,3 and 4) in the culture medium, or conditioned medium, or as substrate coating.
4. activating or inhibiting endogeneous expression of a gene involved in R-spondin factor signaling in said target cells; or,
5. overexpressing proteins involved in controlling R-spondin factor expression level, maturation and overall regulation in said target cells.

In one embodiment, the culture medium of step a) comprises CHIR99021 and an inhibitor of BMP signaling pathway according to the invention which is Dorsomorphin to improve the differentiation of pluripotent cells into induced Paraxial Mesoderm progenitor (iPAM) cells.

In one embodiment, the culture medium of step a) comprises CHIR99021, Dorsomorphin and DMSO to improve the differentiation of pluripotent cells into induced Paraxial Mesoderm progenitor (iPAM) cells.

In one embodiment, the culture medium of step a) comprises a Wnt activator which is a combination of R-spondin2, R-spondin3 and CHIR99021; and an inhibitor of BMP signaling which is a combination of Noggin and Dorsomorphin to improve the differentiation of pluripotent cells into induced Paraxial Mesoderm progenitor (iPAM) cells.

In still another embodiment, the culture medium of step a) comprises R-spondin 3, R-spondin 2, CHIR99021, Dorsomorphin and DMSO to improve the differentiation of pluripotent cells into induced Paraxial Mesoderm progenitor (iPAM) cells.

In still another embodiment, the culture medium of step a) comprises R-spondin 3, R-spondin 2, CHIR99021, Noggin and DMSO to improve the differentiation of pluripotent cells into induced Paraxial Mesoderm progenitor (iPAM) cells.

In still another embodiment, the culture medium of step a) comprises or consists of or consists essentially of CHIR99021 and LDN-193189. Even in another embodiment, the culture medium of step a) comprises or consists of or consists essentially of CHIR99021 (3 μM) and LDN-193189 (500 nM).

In a preferred embodiment, the activator is a member of the R-spondin family.

In another embodiment, the activator is selected from the group consisting of R-spondin 1, R-spondin 2, R-spondin 3 and R-spondin 4.

In another preferred embodiment, the activator is the R-spondin 2 or the R-spondin 3.

In another preferred embodiment, the activator is an inhibitor of GSK-3β such as CHIR99021.

In another embodiment, the inhibitor according to the invention is a secreted antagonist of the BMP/TGFbeta family.

In another embodiment, the inhibitor of BMP signaling pathway is selected from the group consisting of Noggin, Chordin, Chordin-like 1/2/3, Follistatin, Follistatin-like 1/2/3/4/5, a member of the Dan family, including Cerberus 1, Gremlin 1/2.

In another preferred embodiment, the inhibitor is Noggin or Follistatin.

In another preferred embodiment, the inhibitor is a chemical inhibitor of BMP signaling such as Dorsomorphin.

The duration of step a) is not critical as long as an appropriate amount of iPAM cells or an iPAM cell population has been obtained. The duration may also vary depending on the presence of a BMP inhibitor and the presence of DMSO. Usually the duration of step a) may be ranged from 3 and 12 days or from 4 and 11 days or from 5 and 10 days or from 6 and 9 days or may be 3, 4, 5, 6, 7, 8, 9, 10, 11, 12 days.

In an embodiment, pluripotent cells, preferably iPS, more preferably hiPS cells are dissociated to single cells using trypsin and seeded at a density ranging from 3.10⁴ to 9.10⁴ cells/cm². A preferred density is 5.5.10⁴ cells/cm². The culture may be carried out on matrigel-coated dishes in mTESR-1 medium supplemented with Rock-1 inhibitor (10 μM). One day after, medium may be changed to fresh mTESR-1 without Rock-1 inhibitor. The medium (preferably DMEM supplemented with ITS (1%)) may be supplemented with with FGF-2 (20 ng/ml) from day 3. The medium may be refreshed daily until day 6.

The only specific marker for paraxial mesoderm progenitor cells is Msgn1 (Yoon J K et al 2015). The expression of Msgn1 may be assessed as explained in the part entitled definitions to be applied in the context of the application.

As used herein, the Msgn1 gene refers to the gene encoding Mesogenin1. Within the context of the invention, the Msgn1 gene refers to the mammalian gene encoding Msgn1, preferably murine or human gene. Examples of a nucleotide sequence of a gene encoding Mesogenin1 in mouse and human are given in SEQ ID NO:8 (NM_019544.1) and SEQ ID NO:9 (NM_001105569.1) respectively.

In one embodiment, Msgn1 is considered expressed, when expression is detectable in a quantitative assay for gene expression. In another embodiment, Msgn1 is considered to be expressed when the expression level is significantly higher than the expression level observed in the original pluripotent cells, or in cells differentiating under non specific conditions such as Basal culture medium without LIF (Leukemia Inhibitory Factor) for mouse pluripotent cells or without FGF (Fibroblast Growth Factor) for human pluripotent cells. Expression levels between the control and the test cells may be normalized using constitutively expressed genes such as GAPDH or Beta Actin.

Other biomarkers characteristic of paraxial mesoderm progenitor cells include, without limitation, one or more of the following proteins: Tbx6, EphrinA1, EphrinB2, EPHA4, PDGFRalpha, Sall1, Sall4, Dll1, Dll3, Papc (Pcdh8), Lfng, Hes7, Ripplyl, Ripply2, Brachyury (T), Cdx2, Cdx4, Evx1, Cxcr4, III7rd, Fgf8, Fgf17, Gbx2, Wnt3a, Wnt5b, Rspo3, SP5, SP8, Has2, Dkk1, Dact1, Pax3, Pax7, Mesp1, Mesp2.

These iPAM populations typically may comprise other cell types in addition to iPAM cells. In one embodiment, the populations obtained at the end of step a) are characterized in that they comprise at least 10%, 20%, 30%, 40%, 50%, 60%, 70%, 80% and preferably at least 90% of cells that express at least one biomarker characteristic of iPAM cells, for example Msgn1. The assessment of the presence or expression of said marker is preferably carried out as explained in the part entitled definitions to be applied in the context of the invention.

Populations comprising iPAM cells may be cultured indefinitely under appropriate growth conditions .

The iPAM cells may be purified or the populations may be enriched in iPAM cells by selecting cells expressing markers specific of iPAM cells. In one embodiment, markers specific of iPAM cells for purification or enrichment of a population of iPAM cells may be Msgn1.

Purification or iPAM cells enrichment may be achieved using cell sorting technologies, such as fluorescence activated cell sorting (FACS) or magnetic beads comprising specific binders of said cell surface markers of iPAM cells, or fluorescent reporters for iPAM markers.

After purification or enrichment, the population may thus comprise more than 10%, 20%, 30%, 40%, 50%, 60%, 70%, 80%, 90% or more than 95% of cells expressing a biomarker characteristic of iPAM cells, for example, Msgn1.

Step b

IPAM cells or a population comprising iPAM cells obtained at the end of step a) are subsequently cultured in a myogenic culture medium. As indicated above, the cells obtained at the end of step a) may have been first further enriched or purified for the presence of iPAM cells.

A myogenic culture medium as used herein is a culture medium which facilitates, stimulates, induces the production of progenitor of muscle cells and/or muscle cells. A marker of progenitor of muscle cells may be Pax7 (Zammit, et al 2006). A marker of early stage muscle cells may be desmin or myogenin (Paulin et al 2004, Buckingham et al 2014). A marker of mature muscle cells may be alpha actinin (Beggs et al 1992). The expression of each of these markers may be assessed as explained in the part entitled definitions to be applied in the context of the application.

A myogenic culture medium may comprise or consist of or essentially consist of a culture medium, serum or an equivalent thereof, (preferably KSR), an inhibitor of a BMP receptor, an activator of the c-MET receptor and an activator of an IGF or insulin receptor.

A culture medium within the context of the application is a medium suitable for culturing mammalian cells. Suitable culture media are known to the skilled person and include DMEM, RPMI 1640, MEM, Ham's F12, IMDM, Leibovitz medium Medium 199. A preferred culture medium is DMEM.

Serum may be bovine serum. A preferred bovine serum is fetal bovine serum. In another preferred embodiment, an equivalent of serum is KSR (Knockout Serum Replacement) as described in Chal et al 2015. A preferred inhibitor or a BMP receptor is LDN193189. LDN193189 is an inhibitor of the BMP type I receptor Alk2 and Alk3. A preferred activator of the c-MET receptor is HGF (Hepatocyte Growth Factor). A preferred activator of an IGF or insulin receptor is IGF-1 (Insulin Growth Factor-1).

In a first embodiment, a myogenic culture medium comprises or consists of or essentially consists of a culture medium, serum or an equivalent thereof (preferably KSR), an inhibitor of a BMP receptor (preferably LDN193189), an activator of the c-MET receptor (preferably HGF) and an activator of an IGF or insulin receptor (preferably IGF1). A preferred myogenic culture medium in this first embodiment comprises or consists of or essentially consists of a culture medium, KSR, LDN193189, HGF and IGF-1.

In a second embodiment, a myogenic culture medium comprises or consists of or essentially consists of a culture medium, serum or an equivalent thereof (preferably KSR), an activator of the c-MET receptor (preferably HGF) and an activator of an IGF or insulin receptor (preferably IGF1). A preferred myogenic culture medium in this second embodiment comprises a culture medium, KSR, HGF and IGF-1.

In a third embodiment, a myogenic culture medium comprises or consists of or essentially consists of a culture medium, serum or an equivalent thereof (preferably KSR) and an activator of an IGF or insulin receptor (preferably IGF1). A preferred myogenic culture medium in this third embodiment comprises a culture medium, KSR and IGF-1.

In a preferred step b), the myogenic culture medium of the first embodiment is first used, followed by the myogenic culture medium of the third embodiment, subsequently followed by the myogenic culture medium of the second embodiment. The culture in the myogenic culture medium of the first embodiment may have a duration of 1 to 3 days or 2 days. The culture in the myogenic culture medium of the third embodiment may have a duration of 3 to 6 days or 4 to 5 days or 4 days. The culture in the myogenic culture medium of the second embodiment may have a duration of 8 to 12 days or 9 to 11 days or 10 days.

In a preferred method, step b) is carried out using a myogenic culture medium that comprises or consists of or essentially consists of a culture medium, KSR, LDN193189, HGF and IGF-1.

Preferred concentrations are the following: bovine serum or fetal bovine serum from 5 to 10%, KSR from 8 to 20% or from 10 to 18% or from 12 to 16% or from 13 to 16% or from 14 to 16% or 15%. Preferred concentrations of LDN193189 are from 300 to 600 nM or from 400 to 500 nM or about 500 Nm or 500 nM. The concentration of HGF may be from 8 to 12 ng/ml or from 9 to 11 ng/ml or about 10 ng/ml or 10 ng/ml. The concentration of IGF-1 may be from 0.8 to 4 ng/ml or from 1 to 3 ng/ml or from 1.5 to 2.5 ng/ml of about 2 ng/ml or 2 ng/ml.

A preferred myogenic culture medium in this first embodiment comprises or consists of or essentially consists of a culture medium, KSR (15%), LDN193189 (500 nM), HGF (10 ng/ml) and IGF-1 (2 ng/ml).

The duration of step b) is not critical. The duration may also vary depending on the number of iPAM cells used at the onset of step b) and/or of the identity of the components present in the myogenic culture medium. Usually, the duration of step b) may be ranged from 2 and 18 days, 3 and 18 days or from 4 and 17 days or from 5 and 16 days or from 6 and 16 days.

Step c)

This step is optional. This step comprises culturing the cells or the population of cells obtained at the end of step b) in a culture medium comprising serum or an equivalent thereof, and further optionally comprising FGF2 (Fibroblast Growth Factor 2) or an equivalent thereof. FGF2 (Fibroblast Growth Factor 2) is identical with bFGF (basicFibroblast Growth Factor). This step is intended to increase the number of BAP cells present or enrich the number of BAP cells since there is no longer a myogenic culture medium present triggering the cells to differentiate towards the muscle pathway.

Serum may be bovine serum, preferably fetal bovine serum. Serum may be present from 5 to 10%, preferably 10%. An equivalent of serum is KSR.

Optionally in step c) FGF2 or an equivalent thereof may be present. It means that step c) may be carried out without the presence of FGF2.

The concentration of FGF2 may be from 3 to 7 ng/ml or from 4 to 6 ng/ml or about 5 ng/ml or 5 ng/ml. The concentration of KSR may be as in step b).

The duration of step c) is not critical. The duration may also vary depending on the number of myogenic cells used at the onset of step c) and/or of the identity of the components present in the myogenic culture medium of step b) and/or the duration of step b). Usually, the duration of step c) may be ranged from 3 and 15 days or from 4 and 15 days, or from 5 and 15 days or from 6 and 15 days or from 3 and 12 days or from 4 and 11 days or from 5 and 10 days or from 3 and 9 days or from 4 and 8 days or from 5 and 7 days or 6 days. The medium may be refreshed every 2 days.

Step d)

In step d) cells or a population of cells obtained at the end of step b) or c) are further cultured by passaging said cells or said population of cells and seeding them into culture dish. The density used to seed the cells may be ranged from from 3.10⁴ to 9.10⁴ cells/cm² on tissue culture grade plate, preferably the cell density is 5.10⁴ cells/m².

Cells or the population of cells obtained at the end of step b) or c) may already comprise some BAP cells. Step d) is intended to further enrich and expand them. The culture medium of step d) may not comprise any driver of a specific differentiation. The culture medium of step d) may comprise or consist or essentially consist of a medium such as those already listed herein.. DMEM is preferred as it promotes or facilitates proliferation of cells. For example no compound inducing myogenic or adipogenic differentiation may be present in the medium of step d). Usually this medium may comprise DMEM, glucose, serum or an equivalent thereof and FGF2 or an equivalent thereof. In an embodiment, bovine serum is present in said medium. In an embodiment, fetal bovine serum is present in said medium. In an embodiment, KSR is used as an equivalent of serum. In an embodiment, 5 to 10% bovine serum or fetal bovine serum is present in said medium. Concentrations of KSR may be as those defined for step b). In an embodiment, 3 to 8 ng/ml or 4 to 7 or 4 to 6 or 5 ng/ml FGF2 is present in said medium. In an embodiment, glucose is present from 0.5 to 6 g/L, or 0.8 to 5 g/L or 1 to 4.5 g/l. Preferably glucose is present at 1 g/L.

The enrichment and expansion of BAP cells in step d) may be monitored by assessing the homogeneity of the cell population obtained. The homogeneity may be assessed in relation to the morphology of the cells, the proliferative capacity of the cells and/or the expression of a given marker. The preferred morphology of the homogeneous cell population is a fibroblast-like morphology which means spindle-shaped. The morphology could be observed under the microscope.

In addition, in a preferred embodiment, the homogeneous cell population is highly proliferative until reaching 90-100% confluence within 24 to 72 hours. Usually, confluent cells or cells about to be confluent (90-100% confluent) are seeded into culture dishes at a density of 50 000 cells/cm². In a preferred embodiment, a cell population is said to be homogeneous and highly proliferating when confluence is reached within 24 to 72 hours.

The duration of step d) is not critical. The duration may also vary depending on the number of cells used at the onset of step c) and/or of the identity of the components present in the myogenic culture medium. The number of passages during step d) may be ranged from 2 to 10, 3 to 9 or at least 2, 3, 4, 5, 6, 7, 8, 9, 10 passages or at the most 3, 4, 5, 6, 7, 8, 9, 10 passages. In a preferred embodiment, the number of passages is 4 to 9, more preferably at least 4 or 4. Usually each passage has a duration of 2 to 3 days. Usually, the duration of step d) may be ranged from 3 and 27 days or from 4 and 26 days or from 5 and 25 days or from 8 and 12 days. The duration may have to be corrected depending on the number initial cells seeded.

Step d) is intended to expand and enrich for BAP cells by passaging the cells obtained at the end of step b) or c) and seeding them into culture dish. BAP cells or a population of BAP cells is later defined herein.

In a further aspect, the present invention also relates to a method for preparing BA cells, said method comprising the following steps:

Step a), step b), optional step c) and step d) preferably as defined above and further comprising the following step:
e) Culturing selected BAP cells preferably those obtainable at the end of step d) in an adipogenic culture medium comprising serum or an equivalent thereof obtaining BA cells.

Step e)

Cells preferably obtained or obtainable at the end of step d) or a population of cells preferably obtained or obtainable at the end of step d) are further cultured in an adipogenic culture medium for obtaining BA or a population of BA cells

An adipogenic culture medium as used herein is a culture medium which facilitates, stimulates, induces the production of BA cells. BA cells represent a specialized subpopulation of adipocytes. Some adipocytes which are not BA cells may also be present at the end of step e). BA cells are characterized by the expression of UCP1. Adipocytes are characterized by the expression of FABP4. The expression of UCP1 and FABP4 may be assessed as defined in the part of the description entitled Definitions to be applied in the context of the application.

In an embodiment, a method is carried out wherein the adipogenic culture medium of step e) comprises or essentially consists of a culture medium, an inhibitor of the TGFbeta/Activin/NODAL pathway (preferably SB431542), an activator of the EGF (Epidermal Growth Factor) receptor (preferably EGF), ascorbic acid, and an activator of a corticoid receptor (preferably hydrocortisone). Indomethacin may be added in any of the adipogenic culture medium defined herein. This is a commonly used molecule in such a medium. In a preferred embodiment, a method is carried out wherein the adipogenic culture medium of step e) comprises or essentially consists of a culture medium, SB431542, EGF, ascorbic acid, and hydrocortisone.

In a first embodiment, an adipogenic culture medium may comprise, may consist of or may essentially consist of a culture medium, an inhibitor of the TGF-beta/Activin/NODAL pathway (preferably SB431542), an activator of the EGF (Epidermal Growth Factor) receptor (preferably EGF), a PPARgamma activator (preferably Rosiglitazone), insulin, T3 hormone, ascorbic acid, an activator of a corticoid receptor (i.e. preferably hydrocortisone and dexamethasone), and a non-specific inhibitor of cyclic AMP and cyclic AMP phosphodiesterases (preferably IBMX). In a first preferred embodiment, the adipogenic culture medium comprises or consists of or essentially consists of a culture medium, SB431542, EGF, Rosiglitazone, insulin, T3 hormone, ascorbic acid, hydrocortisone, dexamethasone and IBMX.

In a second embodiment, an adipogenic culture medium may comprise, may consist of or may essentially consist of a culture medium, an inhibitor of the TGF-beta/Activin/NODAL pathway (preferably SB431542), an activator of the EGF (Epidermal Growth Factor) receptor (preferably EGF), a PPARgamma activator (preferably Rosiglitazone), insulin, T3 hormone, ascorbic acid and an activator of a corticoid receptor (preferably a glucocorticoid more preferably hydrocortisone). In a second preferred embodiment, the adipogenic culture medium of the second embodiment comprises or consists of or essentially consists of a culture medium, SB431542, EGF, Rosiglitazone, insulin, T3 hormone, ascorbic acid and hydrocortisone.

Within the context of the invention, an inhibitor of the TGF-beta/Activin/NODAL pathway is preferably a compound that inhibits ALK5, ALK4, and ALK7, but preferably does not inhibit the BMP type I receptors ALK2, ALK3, and ALK6. Such a preferred compound is SB431542 from Manufacturer: Stemcell technologies.

Within the context of the invention, a PPARgamma activator may be an anti-diabetic drug from the thiazolidinedione class and is preferably Rosiglitazone (Manufacturer Prestwick). As known to the skilled person, a compound of the thiazolidinedione class acts by activating the intracellular receptor class of the peroxisome proliferator-activated receptors (PPARs), specifically PPARgamma.

Within the context of the invention, a corticoid receptor includes glucocorticoid receptors and mineralocorticoid receptors. Preferred activators of corticoid receptors include glucocorticoid, more preferably hydrocortisone).

A preferred activator of the EGF receptor is EGF, more preferably human EGF. Human EGF is represented by SEQ ID NO:12. In one embodiment, a functional variant of hEGR may be used. A functional variant is a functional homologue of human EGF having at least 60%, 70%, 80%, 90%, 91%, 92%, 93%, 94%, 95%, 96%, 97%, 98%, 99% or 100% identity to human EGF SEQ ID NO:12 and retaining substantially the same EGF receptor activation activity as the related wild type human EGF. In this context substantially the same activation activity may mean at least 50%, 60%, 70%, 80%, 90% or 100%.

Within the context of the invention an adipogenic culture medium of the first and of the second embodiment comprises a culture medium such as DMEM (Dulbecco's Modified Eagle Medium, Gibco) and serum or an equivalent of serum. Preferably the serum present is bovine serum, more preferably fetal bovine serum. Even more preferably 5 to 10% of bovine serum and most preferably 10%. Even more preferably 5 to 10% of fetal bovine serum and most preferably 10% of fetal bovine serum. In an embodiment, if serum is not present, one can replace it by using KSR. Preferred concentrations of KSR have been already defined herein.

In a preferred embodiment of step e), cells are first cultured in an adipogenic culture medium of the first embodiment and subsequently in an adipogenic culture medium of the second embodiment. Usually the culture in the first adipogenic culture medium may have a duration of 2 to 15 days or 3 to 14 days or 4 to 13 days or 2 to 10 days or 2 to 8 days or 3 days. In an embodiment, the duration is 2, 3, 4, 5, 6, 7, 8, 9, 10, 11, 12, 13, 14 days.

Preferred concentrations are the following: SB431542 from 2 to 8 microM or from 3 to 7 microM or from 4 to 6 microM or about 5 microM or 5 microM. Preferred concentrations of ascorbic acid are from 10 to 50 microg/ml or from 12.5 to 40 microg/ml or from 15 to 30 microg/ml or about 25.5 microg/ml or 25.5 microg/ml. The concentration of EGF may be from 8 to 12 ng/ml or from 9 to 11 ng/ml or about 10 ng/ml or 10 ng/ml. The concentration of hydrocortisone may be from 1 to 7 microg/ml or from 2 to 6 microg/ml or from 3 to 5 microg/ml of about 4 microg/ml or 4 microg/ml. The concentration of Rosiglitazone may be from 0.5 microM to 2 microM, or 0.75 to 1.5 microM or 0.9 to 1.2 microM or 1 microM. The concentration of insulin may be from 2 to 15 microg/ml or 5 to 12.5 microg/ml or 8 to 11 microg/ml or 10 microg/ml. The concentration of T3 hormone may be ranged from 100 to 300 μM or 150 to 250 μM or 200 μM. The concentration of dexamethasone may be ranged from 0.5 microM to 2 microM, or 0.75 to 1.5 microM or 0.9 to 1.2 microM or 1 microM. The concentration of IBMX may be ranged from 300 to 700 microM or from 400 to 600 microM or 500 microM.

Preferably the adipogenic culture medium of the first preferred embodiment comprises or consists of or essentially consists of a culture medium, SB431542 (5 μM), EGF (10 ng/ml), Rosiglitazone (1 μM), insulin (10 mg/ml), T3 hormone (0.2 nM), ascorbic acid (25.5 μg/m), hydrocortisone (4 μg/ml), dexamethasone (1 μM) and IBMX (500 μM).

Preferably the adipogenic culture medium of the second preferred embodiment comprises or consists of or essentially consists of a culture medium, SB431542 (5 μM), EGF (10 ng/ml), Rosiglitazone (1 μM), insulin (10 mg/ml), T3 hormone (0.2 nM), ascorbic acid (25.5 μg/ml) and hydrocortisone (4 μg/ml).

The culture medium in each of these preferred adipogenic culture media is preferably DMEM. In addition, the culture medium in each of these preferred adipogenic culture media is supplemented with 10% FBS and low glucose. In the art, the skilled person knows that “low glucose ” or “DMEM low glucose” is 1g/I as opposed to “high glucose” or “DMEM high glucose” being 4 g/I. Cells may be seeded at a density ranged from 3.10⁴ to 9.10⁴ cells/cm²) at the onset of step e), preferably using 5.10⁴ cells/cm².In an embodiment, cells are maintained in derivation medium i.e. a medium composed of DMEM. In an embodiment, said medium is supplemented with FBS (10%) and FGF-2 (5 ng/ml).

The duration of step e) is not critical. The duration may also vary depending on the number of iPAM cells used at the onset of step b) and/or of the identity of the components present in the myogenic culture medium and/or whether step c) has been carried out, the way step d) has been carried out, for example the number of passages during step d). Usually, the duration of step e) may be ranged from 3 and 18 days or from 4 and 17 days or from 5 and 16 days or from 6 and 16 days.

In an embodiment, the method of the invention is such that the BA cells obtained are characterized by the expression of UCP1. BA cells and the expression of UCP1 are further described in the next section of the description. A preferred human UCP1 amino acid sequence is identified herein as SEQ ID NO: 13.

As will be apparent to the skilled person, the method described herein is an ex vivo or in vitro method.

It is also apparent to the skilled person that a method for obtaining BA cells does not per se need to comprise step a), b), optional step c) and step d) of the method for obtaining BAP cells. As long as the skilled person is able to obtain BAP cells, he can apply step e) as defined above and will obtain BA cells.

### BAP, BA Cells or Population of BAP, BA Cells Obtainable from the Methods of the Invention

The invention further relates to BAP, BA cells per se or populations of BAP, BA cells per se preferably obtainable from the method as described above. It is clear to the skilled person that the invention also relates to BAT (Brown Adipocytes Tissue) comprising BAP or BA cells. Throughout the application, when reference is made to a composition comprising BAP or BA cells or a population comprising BAP or BA cells, such a composition or such a population may be considered as a tissue comprising such cells. BAT is a tissue comprising BA and BAP cells.

In an embodiment, BAP cells are mammalian, more preferably human BAP cells. In an embodiment, a population of BAP cells is a population of human BAP cells. In an embodiment, BA cells are mammalian, more preferably human BA cells. In an embodiment, a population of BA cells is a population of human BA cells.

The BA cells or populations of BA cells typically may comprise other cell types in addition to BA cells. In one embodiment, the populations of BA cells of the invention are characterized in that they comprise at least 10%, 20%, 30%, 40%, 50%, 60%, 70%, 80% and preferably at least 90% of cells that express at least one biomarker characteristic of BA cells, for example UCP1 (Uncoupling protein 1). Preferably the marker is UCP1. UCP1 is presumed to be the only marker to be exclusive of the matured BA stage (Nedergaard et al 2001, Canon et al 2004). Other markers that may be expressed are PGC1alpha, FABP4, CIDEA, PLIN1, PPARgamma, EBF2, ZIC2, DIO2 and the concomitant presence of lipid droplets (i.e. at least one lipid droplet). The assessment of the expression of UCP1 and/or PGC1alpha and/or any other markers listed herein may be done as described in the part entitled Definitions to be applied in the context of the application. The detection of lipid droplets may be carried out using neutral lipid stains.

BA cells may be purified or the populations may be enriched in BA cells by selecting cells expressing markers specific of BA cells. In one embodiment, markers specific of BA cells for purification or enrichment of a population of BA cells may be selected among one or more of the following markers: UCP1, optionally in combination with any one of PGC1alpha, FABP4, CIDEA, PLIN1, PPARgamma, EBF2, ZIC2 and/or DI02. BA cells may also be selected for the presence of at least one lipid droplet as explained earlier herein.

Purification or enrichment of BA cells may be achieved using cell sorting technologies, such as fluorescence activated cell sorting (FACS) or magnetic beads comprising specific binders of said cell surface markers of BA cells, or fluorescent reporters for BA markers.

After purification or enrichment, the population may thus comprise more than 10%, 20%, 30%, 40%, 50%, 60%; 70%, 80%, 90% or more than 95% of cells expressing a biomarker characteristic of BA cells, for example, UCP1, optionally in combination with any one of PGC1alpha, FABP4, CIDEA, PLIN1, PPARgamma, EBF2, ZIC2 and/or DIO2. BA cells may also comprise at least one lipid droplet as explained earlier herein.

Another way of characterizing the functionality of the BA cells obtained is to assess their capacity of releasing free glycerol upon treatment with an activator of lipolysis such as forskolin or isoproterenol. Preferably, the treatment is with forskolin. More preferably, the treatment is with forskolin for 24 hours at 10 mM. The increase of free glycerol released is at least 20%, 30%, 40%, 50% or even at least 60% for the treated cells compared to the untreated cells. This free glycerol release is an indication of the lipolysis increase. The method of the invention allows to obtain high yield (at least 25%, 30%, 35%, 40%, 45%, 50%, 55%, 60%, 70%, 80% or even 85%) of adipocytes, preferably brown adipocytes that are functional.

In another preferred embodiment, the invention relates to a composition comprising a population of BA cells obtainable from the method as described above. In an embodiment, a population of BA cells may consist of or may essentially consist of BA cells.

In another aspect, the invention also relates to BAP cells or populations of BAP cells which typically may comprise other cell types in addition to BAP cells. In one embodiment, the populations of BAP cells of the invention are characterized in that said population of BAP cells is obtainable by the method of the invention and comprises at least 10%, 15%, 20%, 25%, 30%, 35%, 40%, 45%, 50%, 55%, 60% 65%, 70%, 75%, 80%, 85%, 90%, 95%, 99% or 100% of cells that have the ability to be converted into a population of BA cells as defined herein. In an embodiment, said conversion is assessed after having cultured the BAP cells or the population of BAP cells in an adipogenic culture medium comprising serum or an equivalent thereof according to step e). Said BA cells express UCP1. Other markers that may be expressed are PGC1alpha, FABP4, CIDEA, PLIN1, PPARgamma, EBF2, ZIC2, DIO2. These BA cells may also be characterized by the concomitant presence of lipid droplets (i.e. at least one lipid droplet). The assessment of the expression of UCP1 and or any other markers lister herein may be done as described in the part entitled Definitions to be applied in the context of the application. The detection of lipid droplets may be carried out using neutral lipid stains.

Populations of BAP cells or comprising BAP cells may be cultured for one to two months under appropriate growth conditions known to the skilled person (Wdziekonski et al 2010) BAP cells may be purified or the populations may be enriched in BAP cells by applying the method of the invention.

In another preferred embodiment, the invention relates to a composition comprising a population of BAP cells obtainable from the method as described above. In an embodiment, a population of BAP cells may consist of or may essentially consist of BAP cells.

### Use of BAP or BA Cells or Populations of BAP or BA Cells

The BAP cells may advantageously be cultured in vitro under differentiation conditions to generate brown adipocytes (BA) as earlier defined herein.

In another embodiment, the invention relates to a composition comprising BAP or BA cell obtainable by a method according to the invention. In an embodiment, this composition is a pharmaceutical composition. BAP or BA cells or a population comprising BAP or BA cells or a composition comprising these cells or population of cells can be used as a medicament. The medicament may be used for treating or preventing any disease or condition linked with BAP or BA cells activity. Examples of such disease or condition include metabolic disease such as obesity-related pathologies, metabolic syndrome, diabetes mellitus, hyperlipidemia, NASH (Non-Alcoholic Steato Hepatitis), Energy balance (intake versus expenditure). Accordingly, the invention also relates to the use of BAP cells or populations comprising BAP cells or a composition comprising these cells or populations of cells for the manufacture of a medicament against a disease as mentioned herein. Another aspect of the invention relates to the use of populations comprising BAP or BA cells as the Populations of the Invention.

The Populations of the Invention may be used in a variety of applications, in particular, in research or therapeutic field. One major therapeutic field of application is cell therapy or regenerative medicine. Regenerative medicine can be used to potentially cure any disease that results from malfunctioning, damaged or failing tissue or cells (i.e. BA or BAP or related thereto) by regenerating the damaged tissues or cells in vivo or in vitro or ex vivo by implantation of a population comprising BAP or BA cells obtained as explained herein.

Therefore, in one aspect, the invention relates to the Populations of the Invention for use as a cell therapy product for implanting into a mammal, for example human patient.

In one specific embodiment, the invention relates to a pharmaceutical composition comprising a population of BA cells obtained according to the invention. In another preferred embodiment, the invention relates to a pharmaceutical composition comprising a population of BA cells including for example at least 10², 10³, 10⁴, 10⁵, 10⁶, 10⁷, 10⁸, or at least 10⁹ cells expressing UCP1. In another embodiment, this composition comprises a pharmaceutically acceptable vehicle.

In an embodiment, BAP cells are further cultured or further co-cultured with various cell types to induce their differentiation toward the BA lineage. In another embodiment, BAP cells are directly grafted into a recipient host.

In another preferred embodiment, the invention relates to a composition comprising the Populations of the Invention. The composition comprising the Population of the Invention may be used in cell therapy or regenerative medicine.

In a further aspect BAP or BA cells or populations of BAP or BA cells could be used for screening purposes. An example is the use of BAP cells or populations of BAP cells to screen for the ability of a compound to induce the proliferation, survival and/or further differentiation of BAP cells into BA cells. Another example is to study the activation of mature BA cells to induce their energy expenditure.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method or a cell population or a composition as defined herein may comprise additional step(s), respectively additional component(s) than the ones specifically identified, said additional step(s) respectively component(s) not altering the unique characteristic of the invention. In addition the verb “to consist” may be replaced by “to consist essentially of” meaning that a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value. All patent and literature references cited in the present specification are hereby incorporated by reference in their entirety.

The following examples are offered for illustrative purposes only, and are not intended to limit the scope of the present invention in any way.

### Definitions to be Applied in the Context of the Application

Marker

Several times in the application, a cell or a cell population is characterized by the expression of a marker. An example of such a marker is Oct4, SOX2, Nanog, SSEA 3 and 4, TRA 1/81, Tbx6, EphrinA1, EphrinB2, EPHA4, PDGFRalpha, Sall1, Sall4, Dll1, Dll3, Papc (Pcdh8), Lfng, Hes7, Ripply1, Ripply2, Brachyury (T), Cdx2, Cdx4, Evx1, Cxcr4, III7rd, Fgf8, Fgf17, Gbx2, Wnt3a, Wnt5b, Rspo3, SP5, SP8, Has2, Dkk1, Dact1, Pax3, Pax7, Mesp1, Mesp2 Msgn1, pax7, desmin, myogenin, alpha actinin, UCP1, PGC1alpha, FABP4, CIDEA, PLIN1, PPARgamma, EBF2, ZIC2, D102.

A cell ora cell population will be said to express a given marker when the expression of said marker can be detected. The detection of said expression of said marker can be carried out using any methods known in the art for measuring gene expression, in particular, quantitative methods such as, real time quantitative PCR or microarrays, or methods using gene reporter expression, said gene reporter comprising Msgn1 promoter as described in the experimental part of WO 2013/030243 or qualitative methods such as immunostaining or cell sorting methods identifying cells exhibiting specific biomarkers, including cell surface markers.

Sequence Identity “Sequence identity” is herein defined as a relationship between two or more nucleic acid (nucleotide, polynucleotide, RNA, DNA) sequences, as determined by comparing the sequences. In the art, “identity” also means the degree of sequence relatedness between nucleic acid sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

As used herein, the percent identity between the two amino-acid sequences is a function of the number of identical positions shared by the sequences (i.e., % identity =# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino-acid sequences can be determined using the algorithm of E. Meyers and W. Miller (Comput. Appl. Biosci., 4:11-17,1988) which has been incorporated into the ALIGN program (version 2.0), using a PAM120 weight residue table, a gap length penalty of 12 and a gap penalty of 4.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

In an embodiment, sequence identity is calculated based on the full length of two given SEQ ID NO or on part thereof. Part thereof preferably means at least 50%, 60%, 70%, 80%, 90%, or 100% of both SEQ ID NO.

Activator

As used herein the term “activator of a specific pathway or molecule” such as “activator of the Wnt signaling pathway” (unless otherwise indicated) denotes a substance that enhances or promotes or activates or upregulates or increases an activity linked or associated with said pathway or molecule. Wnt signaling activity. For example, for the canonical Wnt/β-catenin signaling pathway, this activity can be measured by Wnt reporter activity using established multimers of LEF/TCF binding sites reporters, and/or inhibition of GSK-313, and/or activation of canonical Wnt target genes such as T, Tbx6, Msgn1, or Axin2. An activation of a Wnt signaling activity may therefore be assessed as being an increase of a Wnt of Msgn1 reporter activity (Chat J et al 2015) as identified above. Depending on the pathway or molecule, the skilled person knows an assay specific for an activity of said pathway or molecule and that can be used to assess the activator. Said increase may be of at least 1%, 5% 10%, 15%, 20%, 25%, 30%, 35%, 40%, 45%, 50%, 55%, 60%, 65%, 70%, 75%, 80%, 85%, 90%, 95%, 100% or more compared to a control situation with no activator.

Inhibitor

As used herein the term “inhibitor of a specific pathway or molecule denotes a substance that inhibits, downregulates or decreases an activity linked or associated with said pathway or molecule. Depending on the pathway or molecule, the skilled person knows an assay specific for an activity of said pathway or molecule and that can be used to assess the inhibitor. Said decrease may be of at least 1%, 5% 10%, 15%, 20%, 25%, 30%, 35%, 40%, 45%, 50%, 55%, 60%, 65%, 70%, 75%, 80%, 85%, 90%, 95%, 100% or more compared to a control situation with no inhibitor.

### FIGURES AND TABLES

FIG. 1. Process to derive human brown adipocyte progenitors (hBAP) and generate human brown adipocytes (hBA) from hiPS cells. After generation of induced paraxial mesoderm (iPAM), cells undergo myogenic differentiation. BAP are then derived from these cells and induced to differentiate into brown adipocytes.

FIG. 2. Experimental scheme to derive human brown adipocyte progenitors from hiPSC. From day 0 to day 22 a sequence of differentiation media allows to induce successively iPAM and myogenic lineages from hiPS cells on matrigel-coated culture plate. From day 12 to day 22, cells are dissociated using tryspin and seeded on uncoated culture plate. After several passages, cell population is enriched in BAP.

FIG. 3. BAP derivation from hiPS cells. Phase contrast light pictures of cell population during derivation of BAP. Cells acquire homogeneous morphology throughout successive passages (scale bar=1,000 μm).

FIG. 4. Adipogenic potential of BAP. mRNA were prepared from undifferentiated BAP (Und.) or from BAP having undergone differentiation for 17 days at different passages (P5, P6, P7, P8 and P9). Six adipogenic markers have been analyzed by qPCR.

FIG. 5. Characterization of brown adipocytes. BAP differentiation has been induced for 17 days at different passages (P5, P6, P7, P8 and P9). UCP1, lipid droplets and nuclei are visualized by immunostaining (scale bar=50 μm).

FIG. 6. Process to derive human brown adipocyte progenitors (hBAP) and generate human brown adipocytes (hBA) from hiPS cells. After generation of induced paraxial mesoderm (iPAM), cells undergo myogenic differentiation. BAP are then derived from these cells and induced to differentiate into brown adipocytes.

FIG. 7. Experimental scheme to derive human brown adipocyte progenitors from hiPSC. From day 0 to day 22 a sequence of differentiation media allows to induce successively iPAM and myogenic lineages from hiPS cells on matrigel-coated culture plate. From day 12 to day 22, cells are dissociated using tryspin and seeded on uncoated culture plates. After several passages, cell population is enriched in BAP.

FIG. 8. BAP derivation from hiPS cells. Phase contrast light pictures of cell population during derivation of BAP at day 16. Cells acquire homogeneous morphology throughout successive passages (scale bar=1,000 μm).

FIG. 9. Adipogenic potential of BAP over early passages. mRNA were prepared from undifferentiated BAP (Und.) or from BAP having undergone differentiation for 17 days at different passages (P1, P2, P3, P4 and P5). Six adipogenic markers have been analyzed by qPCR.

FIG. 10. Characterization of brown adipocytes over early passages. Differentiation has been induced for 17 days for BAP derived at day 16 at different passages (P1, P2, P3, P4 and P5). UCP1, lipid droplets and nuclei are visualized by immunostaining . (B) Quantification of the cell population expressing UCP1 and presenting lipid droplets.

FIG. 11. Adipogenic potential of BAP over late passages . mRNA were prepared from undifferentiated BAP (Und.) or from BAP having undergone differentiation for 17 days at different passages (P5, P6, P7, P8 and P9). Six adipogenic markers have been analyzed by qPCR.

FIG. 12. Characterization of brown adipocytes over late passages. BAP differentiation has been induced for 17 days at different passages (P5, P6, P7, P8 and P9). (A) UCP1, lipid droplets and nuclei are visualized by immunostaining. (B) Quantification of the cell population expressing UCP1 and presenting lipid droplets.

FIG. 13. Characterization of brown adipocytes from BAP derived at different endpoints.

BAP were derived from day 12 to day 22 after a culturing step in medium containing serum, passaged 5 times and have undergone adipocyte differentiation for 17 days. (A) UCP1, lipid droplets and nuclei are visualized by immunostaining. (B) Quantification of the cell population expressing UCP1 and presenting lipid droplets. (C) mRNA were prepared from undifferentiated BAP (Und.) or from BAP having undergone differentiation for 17 days (Differentiated) and adipogenic markers have been analyzed by qPCR. (D) BAP derived form day 12 or day 16 were induced to differentiate for 17 days, then lipolysis is stimulated with 10 μM forskolin.

FIG. 14. Effect of the culturing step in medium containing serum. (A)BAP were derived from day 20 with (1) or without (2) a culturing step in medium containing serum (i.e. optional step c)) and have undergone adipocyte differentiation for 17 days. UCP1, lipid droplets and nuclei are visualized by immunostaining.

FIG. 15. Comparison to method 2. hiPS cells were induced toward paraxial mesoderm lineage according to the Method 2 . At day 8, cells are maintained in myogenic medium (a) or two adipogenic media (b and c). mRNA were prepared from day 20 and day 30. BAP and BA are also generated from hiPS cells according to the method 1. mRNA were prepared from undifferentiated BAP (Und.) or from BAP having undergone differentiation for 17 days (Diff.) as described in method 1. Two adipogenic markers have been analyzed by qPCR.

FIG. 16. Comparison to method 3.1. After the induction of paraxial mesoderm, the cells are differentiated using the method 3.1. mRNA were prepared from day 20 and day 30. BAP and BA are also generated from hiPS cells according to the method 1. mRNA were prepared from undifferentiated BAP (Und.) or from BAP having undergone differentiation for 17 days (Diff.) as described in method 1. Two adipogenic markers have been analyzed by qPCR.

FIG. 17. Comparison to method 3.2. After the induction of paraxial mesoderm, the cells are differentiated using the method 3.2. At day 8, cells are maintained in two different adipogenic media (3.2. a and b). BAP and BA are also generated from hiPS cells according to the method 1. mRNA were prepared from undifferentiated BAP (Und.) or from BAP having undergone differentiation for 17 days (Diff.) as described in method 1. Two adipogenic markers have been analyzed by qPCR.

FIG. 18. Comparison to the method 4. BAP were generated from hiPS cells as described in the method 4 or the method 1. (A)BAP differentiation has been induced for 17 days at P5. UCP1, lipid droplets and nuclei are visualized by immunostaining. (B)mRNA were prepared from undifferentiated BAP (Und.) or from BAP having undergone differentiation for 17 days (Diff) at P5. Two adipogenic markers have been analyzed by qPCR.

FIG. 19. Comparison to the method 5. (A) Phase contrast light pictures of cell population during differentiation hiPS cells as described in the method 5.). BAP and BA are also generated from hiPS cells according to the method 1. (B) mRNA were prepared from undifferentiated BAP (Und.) or from BAP having undergone differentiation for 17 days (Diff.) as described in method 1. Two adipogenic markers have been analyzed by qPCR. (C) After 17 days of differentiation, UCP1, lipid droplets and nuclei are visualized by immunofluorescence on BA cultures.

FIG. 20. Summary of the experiments of comparison. hiPS cells are differentiated using the method 2, the method 3.1, 3.2 and the method 4. For each method tested, BAP (Und.) and BA (Diff.) are also generated from hiPS cells according to the method 1. mRNA were prepared from the different conditions and the expression of the brown adipogenic UCP1 has been analyzed by qPCR.

TABLE 1
Sequences of the invention

| | Bank | | |
|---|---|---|---|
| | Reference | | |
| Proteins | number | SEQ ID | Sequences |
| hRspondin3 | NP-116173.2 | SEQ ID | MHLRLISWLF IILNFMEYIG SQNASRGRRQ |
| | (CAI20141.1) | NO: 1 | RRMHPNVSQG CQGGCATCSD YNGCLSCKPR |
| | or | | LFFALERIGM KQIGVCLSSC PSGYYGTRYP |
| | Q9BXY4-1 | | DINKCTKCKA DCDTCFNKNF CTKCKSGFYL |
| | | | HLGKCLDNCP EGLEANNHTM ECVSIVHCEV |
| | | | SEWNPWSPCT KKGKTCGFKR GTETRVREII |
| | | | QHPSAKGNLC PPTNETRKCT VQRKKCQKGE |
| | | | RGKKGRERKR KKPNKGESKE AIPDSKSLES |
| | | | SKEIPEQREN KQQQKKRKVQ DKQKSVSVST VH |
| mRspondin3 | NP-082627.3 | SEQ ID | MHLRLISCFF IILNFMEYIG SQNASRGRRQ |
| | | NO: 2 | RRMHPNVSQG CQGGCATCSD YNGCLSCKPR |
| | | | LFFVLERIGM KQIGVCLSSC PSGYYGTRYP |
| | | | DINKCTKCKV DCDTCFNKNF CTKCKSGFYL |
| | | | HLG KCLDSCP EGLEAN NHTM ECVSIVH CEA |
| | | | SEWSPWSPCM KKGKTCGFKR GTETRVRDIL |
| | | | QHPSAKGNLC PPTSETRTCI VQRKKCSKGE |
| | | | RGKKGRERKR KKLNKEERKE TSSSSDSKGL |
| | | | ESSIETPDQQ ENKERQQQQK RRARDKQQKS |
| | | | VSVSTVH |
| hRspondin2 | NP-848660.3 | SEQ ID | MQFRLFSFAL IILNCMDYSH CQGNRWRRSK |
| | or | NO: 3 | RASYVSNPIC KGCLSCSKDN GCSRCQQKLF |
| | Q6UXX9-1 | | FFLRREGMRQ YGECLHSCPS GYYGHRAPDM |
| | | | NRCARCRIEN CDSCFSKDFC TKCKVGFYLH |
| | | | RGRCFDECPD GFAPLEETME CVEGCEVGHW |
| | | | SEWGTCSRNN RTCGFKWGLE TRTRQIVKKP |
| | | | VKDTILCPTI AESRRCKMTM RHCPGGKRTP |
| | | | KAKEKRNKKK KRKLIERAQE QHSVFLATDR |
| | | | ANQ |
| mRspondin2 | NP-766403.1 | SEQ ID | MRFCLFSFAL IILNCMDYSQ CQGNRWRRNK |
| | | NO: 4 | RASYVSNPIC KGCLSCSKDN GCSRCQQKLF |
| | | | FFLRREGMRQ YGECLHSCPS GYYGHRAPDM |
| | | | NRCARCRIEN CDSCFSKDFC TKCKVGFYLH |
| | | | RGRCFDECPD GFAPLDETME CVEGCEVGHW |
| | | | SEWGTCSRNN RTCGFKWGLE TRTRQIVKKP |
| | | | AKDTIPCPTI AESRRCKMAM RHCPGGKRTP |
| | | | KAKEKRNKKK RRKLIERAQE QHSVFLATDR |
| | | | VNQ |
| hRspondin3 | CAI20142.1 | SEQ ID | MHLRLISWLF IILNFMEYIG SQNASRGRRQ |
| isoform2 | or | NO: 5 | RRMHPNVSQG CQGGCATCSD YNGCLSCKPR |
| | Q9BXY4-2 | | LFFALERIGM KQIGVCLSSC PSGYYGTRYP |
| | | | DINKCTKCKA DCDTCFNKNF CTKCKSGFYL |
| | | | HLGKCLDNCP EGLEANNHTM ECVSIVHCEV |
| | | | SEWNPWSPCT KKGKTCGFKR GTETRVREII |
| | | | QHPSAKGNLC PPTNETRKCT VQRKKCQKGE |
| | | | RGKKGRERKR KKPNKGESKE AIPDSKSLES |
| | | | SKEIPEQREN KQQQKKRKVQ DKQKSGIEVT |
| | | | LAEGLTSVSQ RTQPTPCRRR YL |
| hRspondin2 | Q6UXX9.2 | SEQ ID | MRQYGECLHS CPSGYYGHRA PDMNRCARCR |
| isoform2 | | NO: 6 | IENCDSCFSK DFCTKCKVGF YLHRGRCFDE |
| | | | CPDGFAPLEE TMECVEGCEV GHWSEWGTCS |
| | | | RNNRTCGFKW GLETRTRQIV KKPVKDTILC |
| | | | PTIAESRRCK MTMRHCPGGK RTPKAKEKRN |
| | | | KKKKRKLIER AQEQHSVFLA TDRANQ |
| hRspondin2 | Q6UXX9-3 | SEQ ID | FRLFSFAL IILNCMDYSH CQGNRWRRSK |
| isoform3 | | NO: 7 | RGCRIENCDS CFSKDFCTKC KVGFYLHRGR |
| | | | CFDECPDGFA PLEETMECVG CEVGHWSEWG |
| | | | TCSRNNRTCG FKWGLETRTR QIVKKPVKDT |
| | | | ILCPTIAESR RCKMTMRHCP GGKRTPKAKE |
| | | | KRNKKKKRKL IERAQEQHSV FLATDRANQ |
| mMsgn1 | NM.019544.1 | SEQ ID | ATGGACAACC TGGGTGAGAC CTTCCTCAGC |
| | | NO :8 | CTGGAGGATG GCCTGGACTC TTCTGACACC |
| | | | GCTGGTCTGC TGGCCTCCTG GGACTGGAAA |
| | | | AGCAGAGCCA GGCCCTTGGA GCTGGTCCAG |
| | | | GAGTCCCCCA CTCAAAGCCT CTCCCCAGCT |
| | | | CCTTCTCTGG AGTCCTACTC TGAGGTCGCA |
| | | | CTGCCCTGCG GGCACAGTGG GGCCAGCACA |
| | | | GGAGGCAGCG ATGGCTACGG CAGTCACGAG |
| | | | GCTGCCGGCT TAGTCGAGCT GGATTACAGC |
| | | | ATGTTGGCTT TTCAACCTCC CTATCTACAC |
| | | | ACTGCTGGTG GCCTCAAAGG CCAGAAAGGC |
| | | | AGCAAAGTCA AGATGTCTGT CCAGCGGAGA |
| | | | CGGAAGGCCA GCGAGAGAGA GAAACTCAGG |
| | | | ATGCGGACCT TAG CCGATGC CCTCCACACG |
| | | | CTCCGGAATT ACCTGCCGCC TGTCTACAGC |
| | | | CAGAGAGGCC AACCGCTCAC CAAGATCCAG |
| | | | ACACTCAAGT ACACCATCAA GTACATCGGG |
| | | | GAACTCACAG ACCTCCTCAA CAGCAGCGGG |
| | | | AGAGAGCCCA GGCCACAGAG TGTGTGA |
| hMsgn1 | NM001105569.1 | SEQ ID | ATGGACAACC TGCGCGAGAC TTTCCTCAGC |
| | | NO: 9 | CTCGAGGATG GCTTGGGCTC CTCTGACAGC |
| | | | CCTGGCCTGC TGTCTTCCTG GGACTGGAAG |
| | | | GACAGGGCAG GGCCCTTTGA GCTGAATCAG |
| | | | GCCTCCCCCT CTCAGAGCCT TTCCCCGGCT |
| | | | CCATCGCTGG AATCCTATTC TTCTTCTCCC |
| | | | TGTCCAGCTG TGGCTGGGCT GCCCTGTGAG |
| | | | CACGGCGGGG CCAGCAGTGG GGGCAGCGAA |
| | | | GGCTGCAGTG TCGGTGGGGC CAGTGGCCTG |
| | | | GTAGAGGTGG ACTACAATAT GTTAGCTTTC |
| | | | CAGCCCACCC ACCTTCAGGG CGGTGGTGGC |
| | | | CCCAAGGCCC AGAAGGGCAC CAAAGTCAGG |
| | | | ATGTCTGTCC AGCGGAGGCG GAAAGCCAGC |
| | | | GAGAGGGAGA AGCTCAGGAT GAGGACCTTG |
| | | | GCAGATGCCC TGCACACCCT CCGGAATTAC |
| | | | CTGCCACCTG TCTACAGCCA GAGAGGCCAG |
| | | | CCTCTCACCA AGATCCAGAC ACTCAAGTAC |
| | | | ACCATCAAGT ACATCGGGGA ACTCACAGAC |
| | | | CTCCTTAACC GCGGCAGAGA GCCCAGAGCC |
| | | | CAGAGCGCGT GA |
| mNoggin | NP_032737 | SEQ ID | MERCPSLGVT LYALVVVLGL RAAPAGGQHY |
| | | NO: 10 | LHIRPAPSDN LPLVDLIEHP DPIFDPKEKD |
| | | | LNETLLRSLL GGHYDPGFMA TSPPEDRPGG |
| | | | GGGPAGGAED LAELDQLLRQ RPSGAMPSEI |
| | | | KGLEFSEGLA QGKKQRLSKK LRRKLQMWLW |
| | | | SQTFCPVLYA WNDLGSRFWP RYVKVGSCFS |
| | | | KRSCSVPEGM VCKPSKSVHL TVLRWRCQRR |
| | | | GGQRCGWIP I QYPI ISECKC SC |
| hNoggin | EAW94528 | SEQ ID | MERCPSLGVT LYALVVVLGL RATPAGGQHY |
| | | NO: 11 | LHIRPAPSDN LPLVDLIEHP DPIFDPKEKD |
| | | | LNETLLRSLL GGHYDPGFMA TSPPEDRPGG |
| | | | GGGAAGGAED LAELDQLLRQ RPSGAMPSEI |
| | | | KGLEFSEGLA QGKKQRLSKK LRRKLQMWLW |
| | | | SQTFCPVLYA WNDLGSRFWP RYVKVGSCFS |
| | | | KRSCSVPEGM VCKPSKSVHL TVLRWRCQRR |
| | | | GGQRCGWIPI QYPI ISECKC SC |
| hEGF | Q6QBS2 | SEQ ID | NSDSECPLSH DGYCLHDGVC MYIEALDKYA |
| (fragment) | | NO: 12 | CNCVVGYIGE RCQYRDLKWWELR |
| hUCP1 | P25874 | SEQ ID | MGGLTASDVH PTLGVQLFSA GIAACLADVI |
| | | NO: 13 | TFPLDTAKVR LQVQGECPTS SVIRYKGVLG |
| | | | TITAVVKTEG RMKLYSGLPA GLQRQISSAS |
| | | | LRIGLYDTVQ EFLTAGKETA PSLGSKILAG |
| | | | LTTGGVAVFI GQPTEVVKVR LQAQSHLHGI |
| | | | KPRYTGTYNA YRIIATTEGL TGLWKGTTPN |
| | | | LMRSVIINCT ELVTYDLMKE AFVKNNILAD |
| | | | DVPCHLVSAL IAGFCATAMS SPVDVVKTRF |
| | | | INSPPGQYKS VPNCAMKVFT NEGPTAFFKG |
| | | | LVPSFLRLGS WNVIMFVCFE QLKRELSKSR |
| | | | QTMDCAT |

### EXAMPLES

### Example 1

Methods

The full names and manufacturer of all compounds used herein is detailed in the Appendix

Primary Differentiation and Derivation of Human Brown Adipocyte Progenitors (hBAP) from hiPS Cells:

Undifferentiated hiPS cells are dissociated to single cells using trypsin and they are seeded at a density of 5.5.10⁴ cells/cm² on matrigel-coated dishes in mTESR-1 medium supplemented with Rock-1 inhibitor (10 μM). One day after, medium is changed to fresh mTESR-1 without Rock-1 inhibitor. When the cells form small aggregates, determined at the day 0 of differentiation, they are changed to a sequence of differentiation media.

At day 0, medium is changed to medium composed of DMEM supplemented with ITS (1%), CHIR99021 (3 μM) and LDN-193189 (500 nM). The medium is refreshed daily until day 6.

At day 6, medium is changed to medium composed of DMEM supplemented with KSR (15%), LDN-193189 (500 nM), HGF (10 ng/ml) and IGF-1 (2 ng/ml). The medium is changed daily until day 8.

At day 8, medium is changed to medium composed of DMEM supplemented with KSR (15%) and IGF-1 (2 ng/ml). The medium is changed daily until day 12.

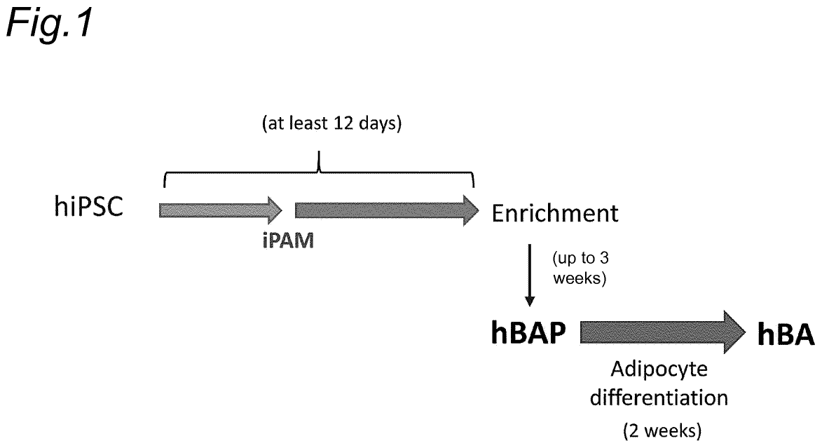

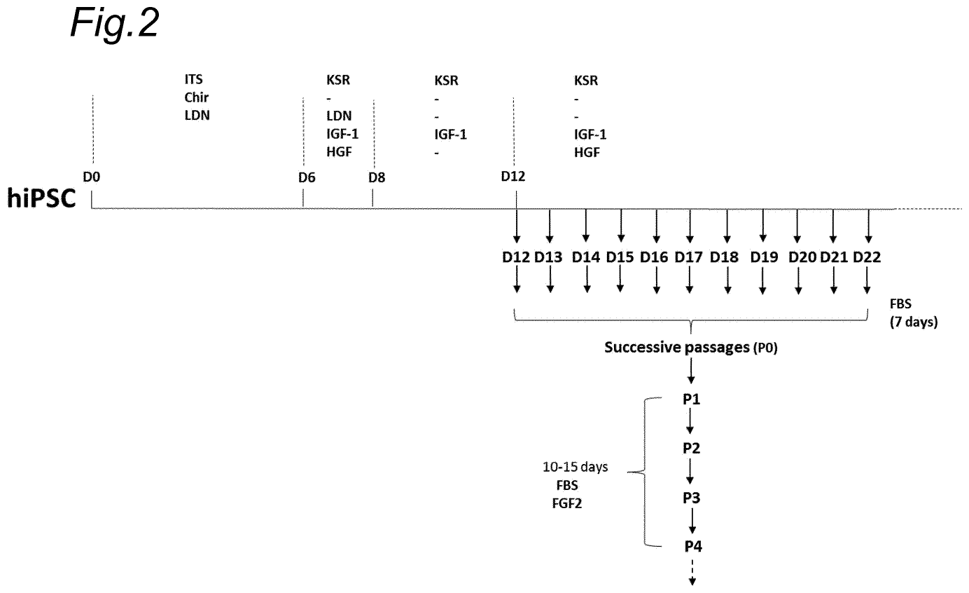

From day 12 to day 22, medium is changed to medium composed of DMEM supplemented with KSR (15%), HGF (10 ng/ml) and IGF-1 (2 ng/ml). The medium is changed every 2 days (FIGS. 1 and 2).

At the selected day to derive hBAP (between day 12 and day 22), medium is changed to a medium composed of DMEM supplemented with FBS (10%). The medium is refreshed every 2 days. After a week, the cells are passaged using trypsin and they're seeded on tissue culture grade plate. This is fixed passage number 0. Cells are maintained in the previous medium supplemented with FGF-2 (5 ng/ml).

When the cells reach confluence, they are passaged and seeded at a density of 5.10⁴ cells/cm².

Passages are repeated (usually 4 to 9 times) until a cell population with homogeneous morphology is obtained (FIG. 2).

Differentiation of Human Brown Adipocyte Progenitors (hBAP):

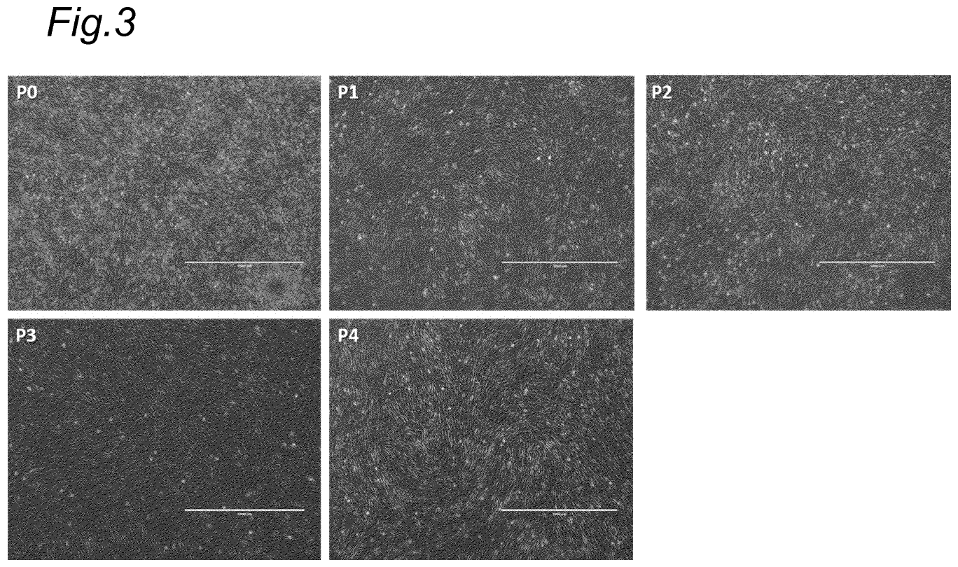

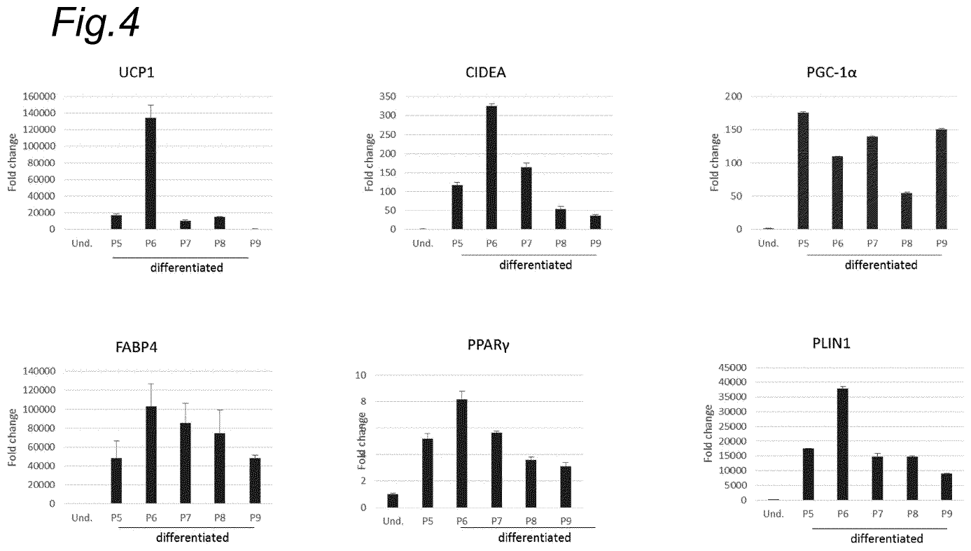

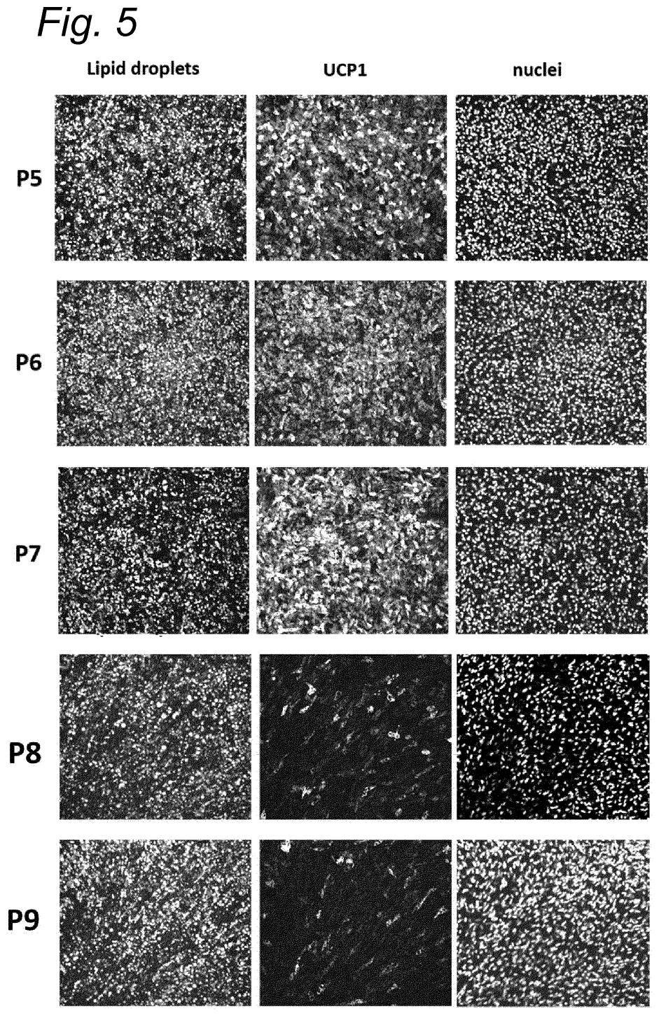

The hBAP are plated at a high density (i.e. 5.10⁵ cells/cm²) and maintained in derivation medium. When the cells reach confluence, determined at the day 0 of differentiation, the medium is changed to differentiation medium composed of DMEM with low glucose supplemented with FBS (10%), rosiglitazone (1 μM), insulin (10 pg/ml), T3 (0.2 nM), SB431542 (5 μM), ascorbic acid (25.5 pg/ml), EGF (10 ng/ml), hydrocortisone (4 μg/ml), dexamethasone (1 μM) and IBMX (500 μM). Dexamethasone and IBMX are discarded after day 3 (FIG. 3). The differentiation medium is then changed twice a week.

Results

Experimental Results

Workflow to get BA.

This protocol is made of sequential differentiation sessions to obtain BA from the initial batch of hiPS cells (FIG. 1). After induction of the iPAM cells, a putative subpopulation of BAP is enriched by sequential replating from passage 5 up to passage 9. BAP were picked at several end points ranging from day 12 to day 22 before starting the second part of the protocol. The enriched BAP population is then differentiated for 2 weeks in a culture media containing key adipogenic factors (FIGS. 1 and 2).

BAP Can Be Enriched Over Passages

After 12 to 22 days of differentiation (day 16 showed), we passaged the cells and plated them in a serum+FGF2 containing culture media to enrich the BAP population. Passaging the cells from P0 to P4, enabled to eliminate aggregates and contaminating cells (FIG. 3). Cell reached an homogeneous and 100% confluent population of fibroblast-like BAP as illustrated by phase-contrast microscopy. After passage 4, the cellular shape of the BAP did not change anymore until P9 (data not shown), allowing the differentiation process indifferently from P5 to P9.

BAP-Derived BA Express Relevant Markers of Differentiated Brown Adipocytes

BAP or the BAP-derived BA (from P5 to P9) were differentiated in DMEM containing 10% Fcetal bovine serum+rosiglitazone (1 μM) +insulin (10 μg/ml) +T3 hormone (200 μM) +SB431542 (5 μm)+Ascorbic acid (25.5 μg/ml)+EGF (10 ng/ml)+hydrocortisone (4 μg/ml)+dexamethasone (1 μM)+IBMX (500 μM). After 3 days, medium was replaced by fresh one lacking the two last compounds. The levels of expression of transcripts by RT-qPCR were assessed on BA and BAP cells (FIG. 4). The pan-adipocyte markers FABP4 (fatty acid binding protein 4), PLIN1 (perilipin 1), PPARγ (peroxisome proliferator-activated receptor gamma) increased into BA cells compared to BAP, reflecting the commitment to adipocyte pathway. On the other hand, brown adipocyte markers: UCP1 (uncoupling protein 1), CIDE-A (cell death-inducing DFFA-like Effector A) and PGC1-α (peroxisome proliferator-activated receptor gamma coactivator alpha) were more expressed in BA than in BAP. This effect was observed from passages 5 to 9 (FIG. 4).

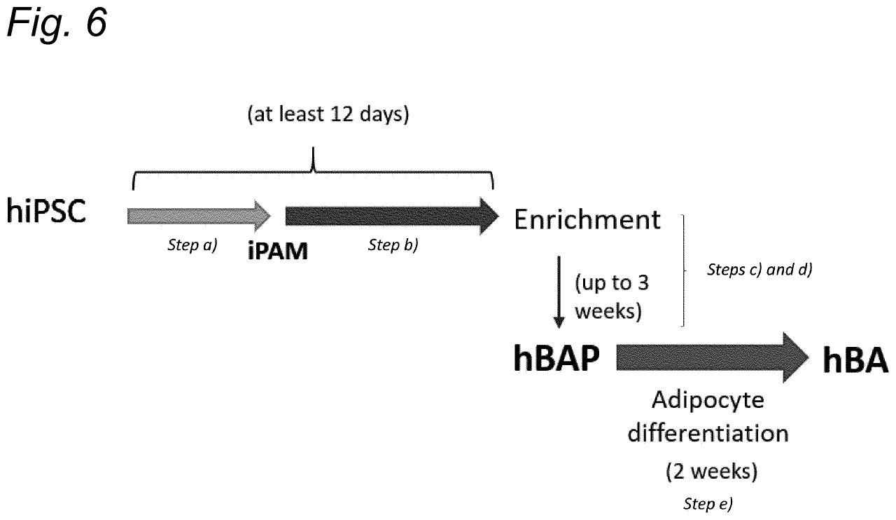

After 17 days of maturation, BA cultures were next characterized by immunofluorescence with an antibody against UCP1 and with a neutral lipid probe identifying the intracellular lipid droplets. As shown, the BA harboured a strong and homogeneous staining for UCP1 (FIG. 6). The quantification of the number of cells expressing UCP1+ lipid droplets showed a range of 39,5% (for P9 cells) to 79% (for P6 cells) positive cells.

### Example 1.a

Methods

The full names and manufacturers of all compounds used herein are detailed in the Appendix

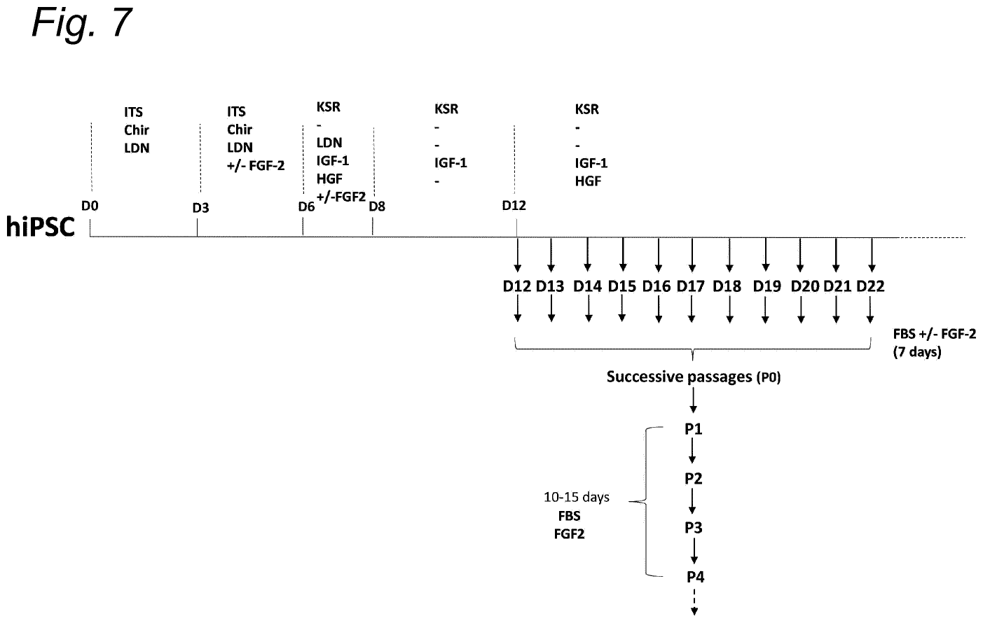

Primary Differentiation and Derivation of Human Brown Adipocyte Progenitors (hBAP) From hiPS Cells (FIGS. 6 and 7):

Step a) Undifferentiated hiPS cells are dissociated to single cells using trypsin and seeded at a density ranging from 3.10⁴ to 9.10⁴ cells/cm². This example was carried out with 5.5.10⁴ cells/cm² on matrigel-coated dishes in mTESR-1 medium supplemented with Rock-1 inhibitor (10 μM). One day after, medium is changed to fresh mTESR-1 without Rock-1 inhibitor. When the cells form small aggregates, determined at the day 0 of differentiation, they are changed to a sequence of primary differentiation media.

At day 0, medium is changed to medium composed of DMEM supplemented with ITS (1%), CHIR99021 (3 μM) and LDN-193189 (500 nM), supplemented or not with FGF-2 (20 ng/ml) from day 3. The medium is refreshed daily until day 6. This corresponds to the induction of the paraxial mesoderm lineage.

Step b) At day 6, medium is changed to medium composed of DMEM supplemented with KSR (15%), LDN-193189 (500 nM), HGF (10 ng/ml) and IGF-1 (2 ng/ml), supplemented or not with FGF-2 (20 ng/ml). The medium is changed daily until day 8.

At day 8, medium is changed to medium composed of DMEM supplemented with KSR (15%) and IGF-1 (2 ng/ml). The medium is changed daily until day 12.

Starting at day 12 and until the selected timepoint is reached (at the latest on day 22), medium is changed to a medium composed of DMEM supplemented with KSR (15%), HGF (10 ng/ml) and IGF-1 (2 ng/ml). The medium is changed every 2 days. This step corresponds to the initiation of the derivation of cells into hBAP.

Step c) On the day selected to derive hBAP (between day 12 and day 22 of the primary differentiation or after 6 to 15 days after initiation of step c)), medium is changed a medium composed of DMEM supplemented or not with FBS (10%), with or without FGF-2 (5 ng/ml).This example was carried out with DMEM supplemented with 10% FBS without FGF-2. The medium is refreshed every 2 days.

Step d) After 7 days, the cells are passaged using trypsin and seeded on tissue culture grade plate. This is passage number 0 (P0). Cells are maintained in a medium composed of DMEM and FBS (10%) supplemented with FGF-2 (5 ng/ml).

When the cells reach confluence, they are passaged and seeded at a density ranging from 3.10⁴ to 9.10⁴ cells/cm² on tissue culture grade plate. This experiment has been carried out using 5.10⁴ cells/cm².

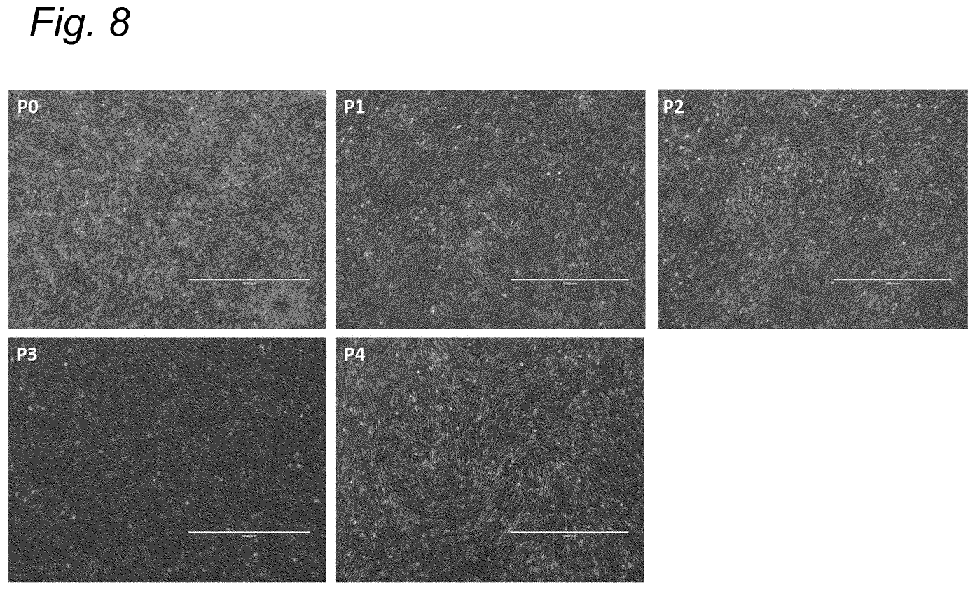

Passages are repeated (usually 4 to 9 times) until a cell population with homogeneous morphology as determined by a person skilled in the art is obtained (FIG. 8), thus generating a population of hBAP.

Secondary Differentiation of Human Brown Adipocyte Progenitors (hBAP) Into Human Brown Adipocytes (hBA):

Step e) The hBAP are plated at a density ranging from 3.10⁴ to 9.10⁴ cells/cm²) and maintained in derivation medium i.e. a medium composed of DMEM supplemented with FBS (10%) and FGF-2 (5 ng/ml). this experiment has been carried out using 5.10⁴ cells/cm². When the cells reach confluence, determined at the day 0 of secondary differentiation, the medium is changed to differentiation medium composed of DMEM with low glucose (1 g/l) supplemented with FBS (10%), rosiglitazone (1 μM), insulin (10 μg/ml), T3 (0.2 nM), SB431542 (5 μM), ascorbic acid (25.5 μg/ml), EGF (10 ng/ml), hydrocortisone (4 μg/ml), dexamethasone (1 μM) and IBMX (500 μM). Dexamethasone and IBMX are discarded after day 3 The differentiation medium is then changed twice a week during 8 to 30 days (usually about 2 weeks).

Quantitative RT-PCR:

Total RNA was extracted from cell cultures using the nucleo spin RNA plus kit (Macherey-Nagel). RT-PCR was performed on 500 ng total RNA using iScript gDNA clear cDNA synthesis Kit (Biorad), appropriate primers and run on a LightCycler 48011 (Roche). TBP was used as the internal control.

Immunocytochemistry:

Cell cultures were fixed with PFA 4%. Cells were incubated for 30 minutes with a blocking solution composed of 5%NGS, 1% fetal bovine serum and 0.2%Triton in Phosphate Buffered Saline (PBS). Primary antibodies incubation was performed during 1 h30 at room temperature and antibodies working dilutions were as follow: anti-UCP1 (R&D) was 1:250, anti-Desmin (Santa Cruz) was 1:800. After PBS washing, cells were incubated with AlexaFluor488-conjugated secondary antibodies (Invitrogen) at 1:1000 for 30 minutes, and counterstained with Dapi. HCS lipidtox neutral lipid staining was done according to standard protocol.

Lipolysis:

The BAP were differentiated for 17 days. Then the cells were maintained for 24 hours in DMEM 1g/Iglucose supplemented with BSA 0,2%. Lipolysis was stimulated with forskolin (10 μM) for 24 hours.

Experimental Results

Workflow to Obtain BA as Described in the Present Invention.

This protocol is made of sequential differentiation sessions to obtain BA from the initial batch of hiPS cells (FIG. 6). After induction of the iPAM cells (step a)) and subsequent exposure to a myogenic medium (step b)), a putative subpopulation of BAP is derived at different timepoints ranging from day 12 to day 22 to be enriched by sequential replating (usually 4 to 9 passages) (steps c) and d)).. This enriched BAP population is then differentiated for about 2 weeks in a culture media containing key adipogenic factors (step e)) (FIGS. 6 and 7).

Phenotypic Characterization of BAP

After 12 to 22 days of differentiation (day 16 showed) according to the method described in this example, cells were plated in a culture media containing FBS+FGF2 to enrich the BAP population. Passaging the cells several times (usually 4) allowed the elimination of aggregates and contaminating cells (FIG. 8). The cell population reached a 100% confluent state of fibroblast-like BAP, homogeneous to a person skilled in the art, as illustrated by phase-contrast microscopy. Additional passages once homogenization is obtained did not change the cellular shape of the BAP (data not shown).

Characterization of BAP-Derived BA Over Passages

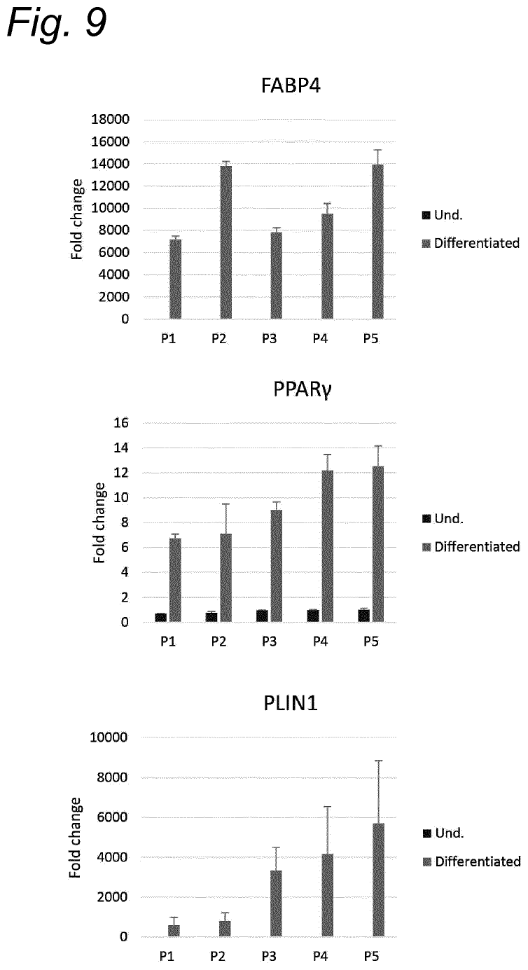

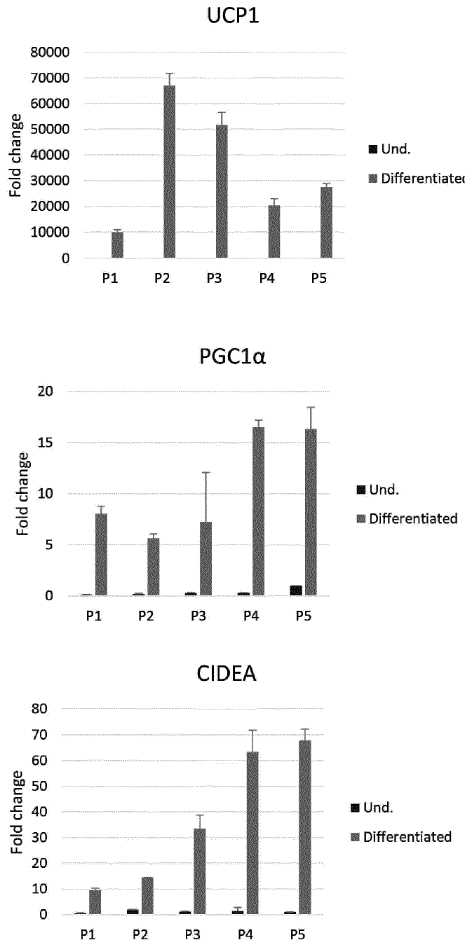

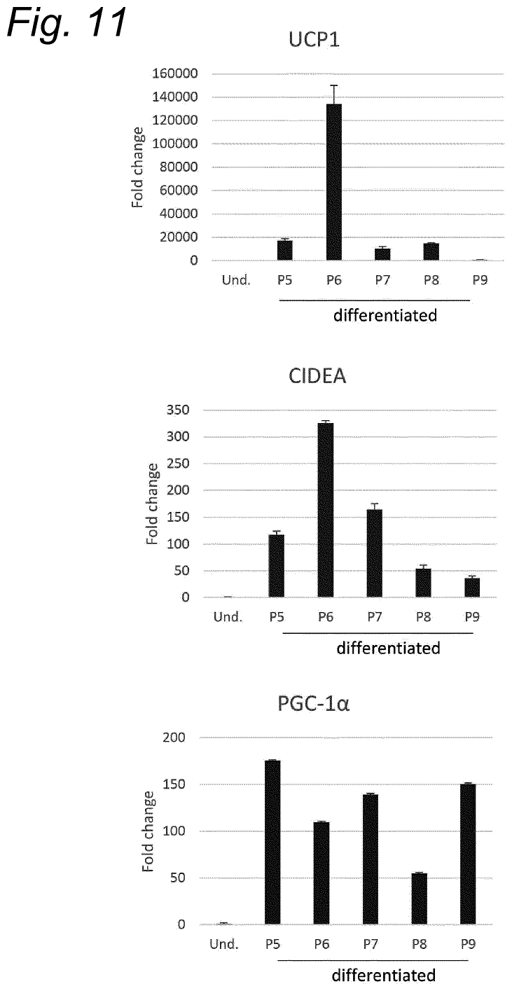

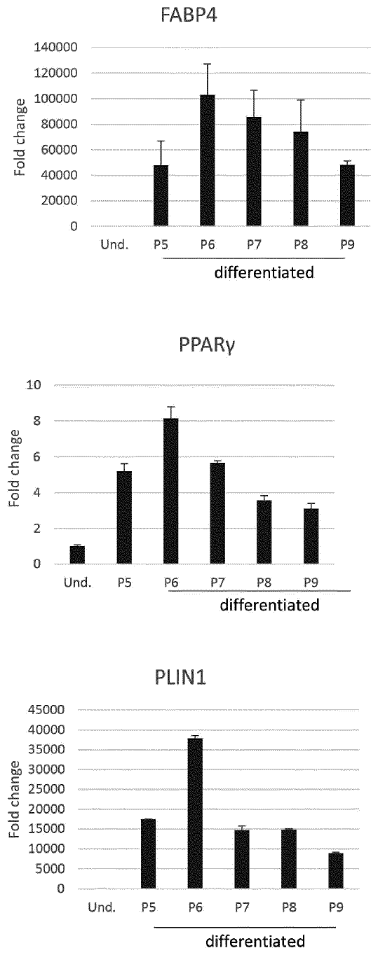

BAP (from P1 to P9) were differentiated into BA in DMEM containing FBS (10%)+rosiglitazone (1 μM)+insulin (10 μg/ml)+T3 hormone (200 μM)+SB431542 (5 μm)+Ascorbic acid (25.5 μg/ml)+EGF (10 ng/ml)+hydrocortisone (4 μg/ml)+dexamethasone (1 μM)+IBMX (500 μM). Dexamethasone and IBMX were discarded after day 3. The levels of expression of certain transcripts typical of the adipocyte lineage were assessed by RT-qPCR both on BAP and BA (FIGS. 9 and 11). The pan-adipocyte markers FABP4 (fatty acid binding protein 4), PLIN1 (perilipin 1), PPARγ (peroxisome proliferator-activated receptor gamma) greatly increased in BA cells compared to BAP (respectively up to 1.10⁵; 3,5.10⁴ and 8 times higher expressed), reflecting the commitment to adipocyte pathway. In addition, brown adipocyte markers: UCP1 (uncoupling protein 1), CIDE-A (cell death-inducing DFFA-like Effector A) and PGC1-a (peroxisome proliferator-activated receptor gamma coactivator alpha) were significantly more expressed in BA than in BAP (respectively up to 1,2.10⁵, 300 and 150 times higher expressed).

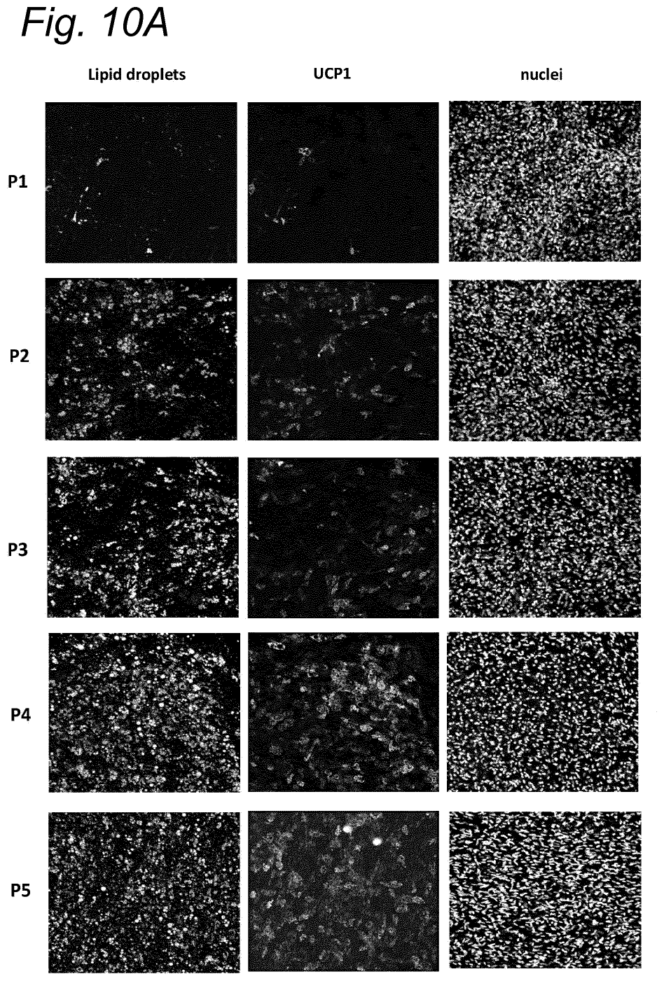

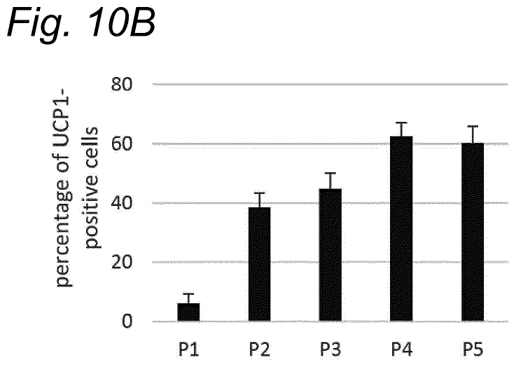

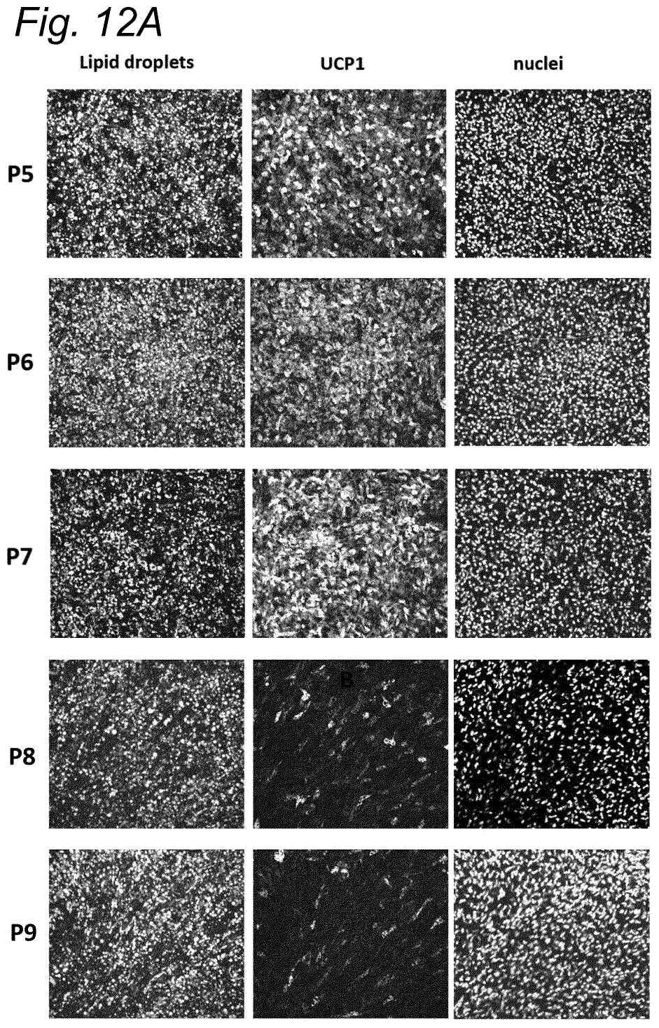

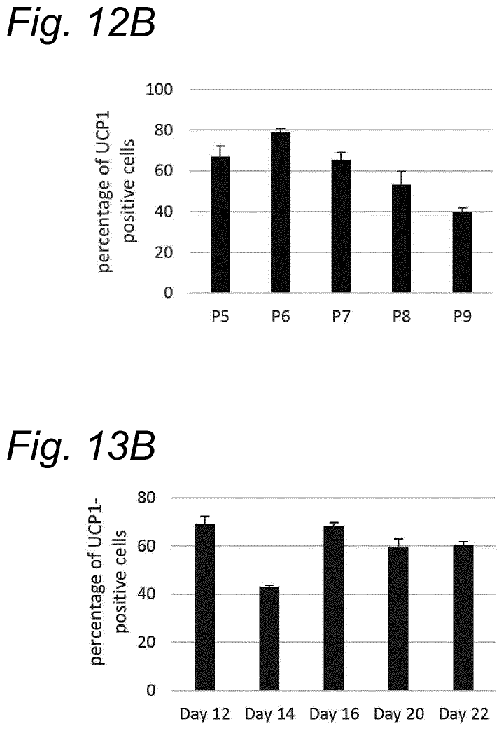

BA cultures at day 17 of differentiation were next characterized by immunofluorescence with an antibody against UCP1 and with a neutral lipid probe identifying the intracellular lipid droplets. The BA harboured a strong and homogeneous staining for UCP1 (FIGS. 10 and 12). The quantification of the number of cells expressing UCP1+ lipid droplets showed a range of 40% (for P2 cells) to 79% (for P6 cells) positive cells for this experiment. The yield of differentiation obtained using the present invention reached 85% of cells expressing UCP1 and presenting lipid droplets (FIG. 19).

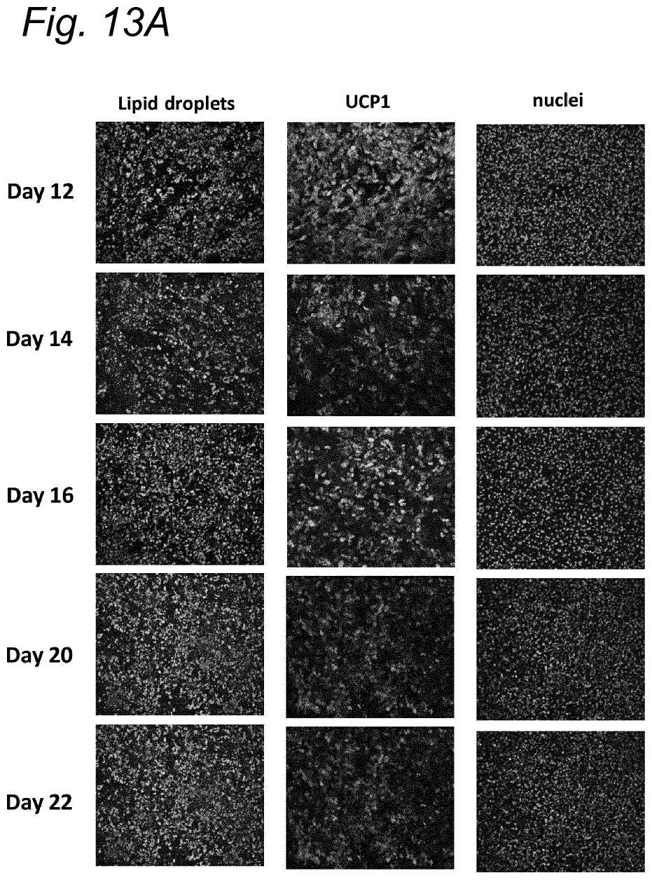

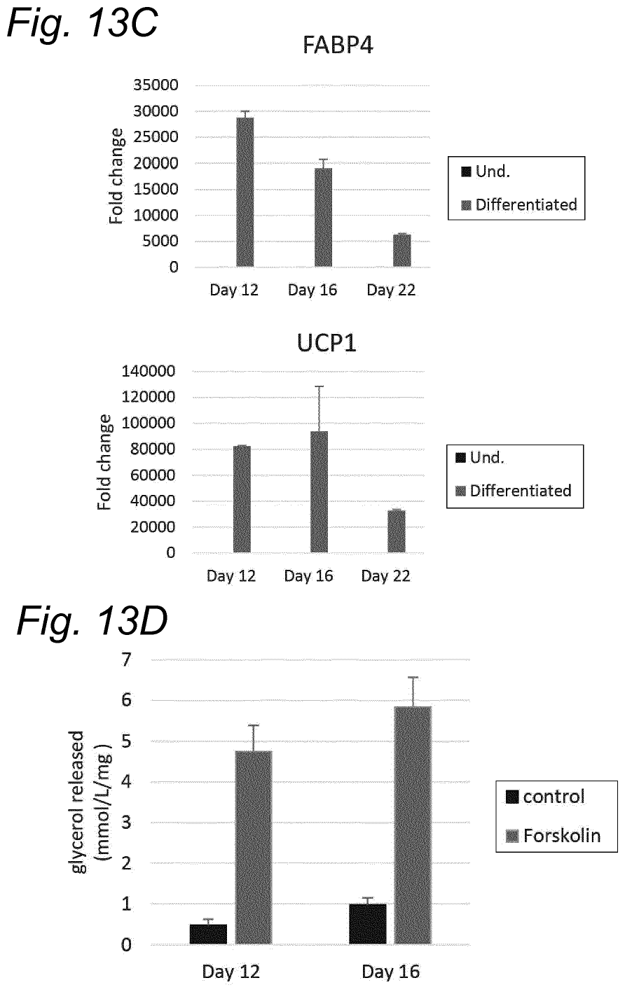

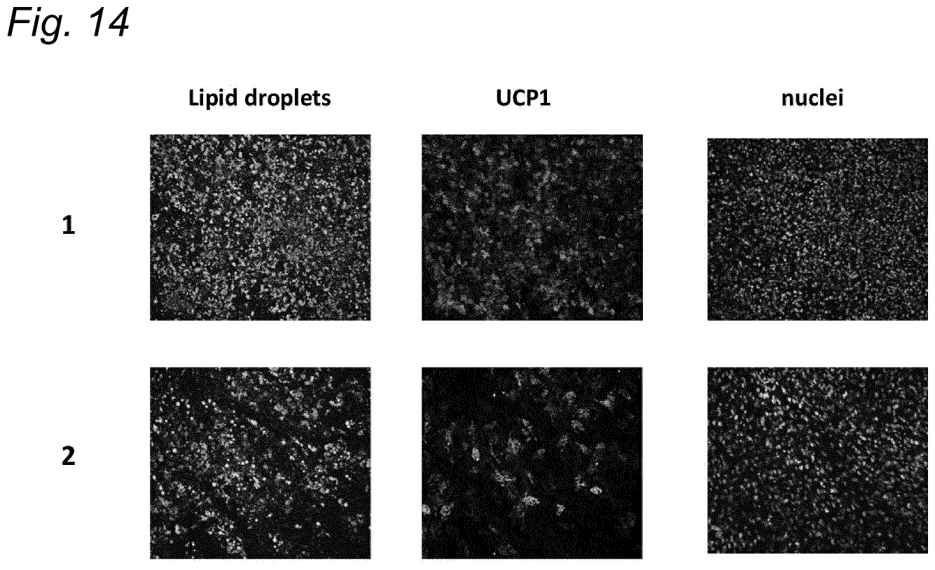

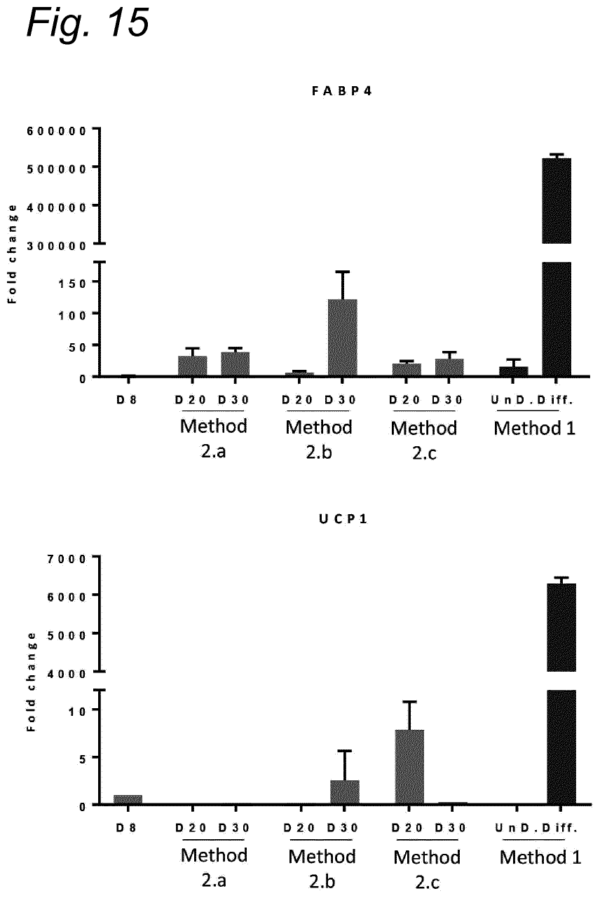

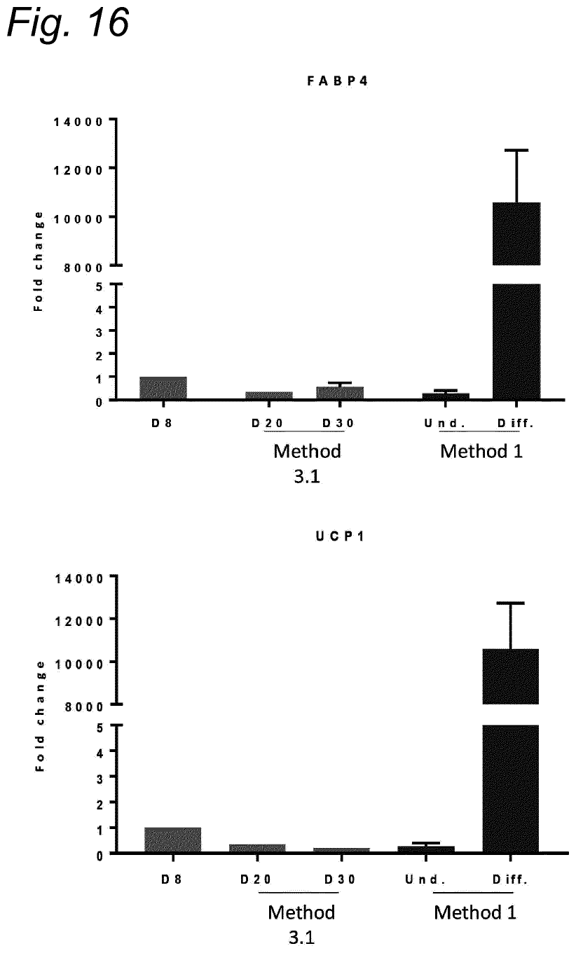

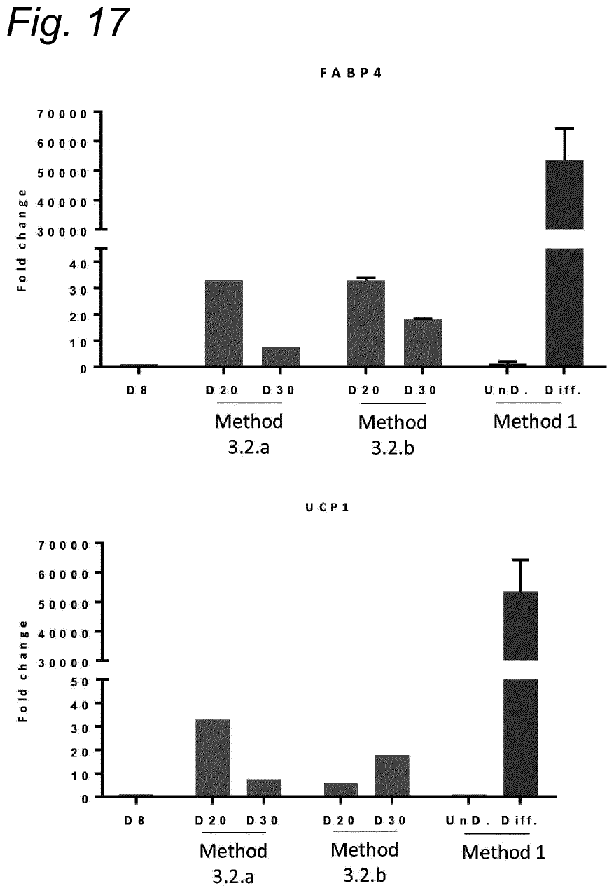

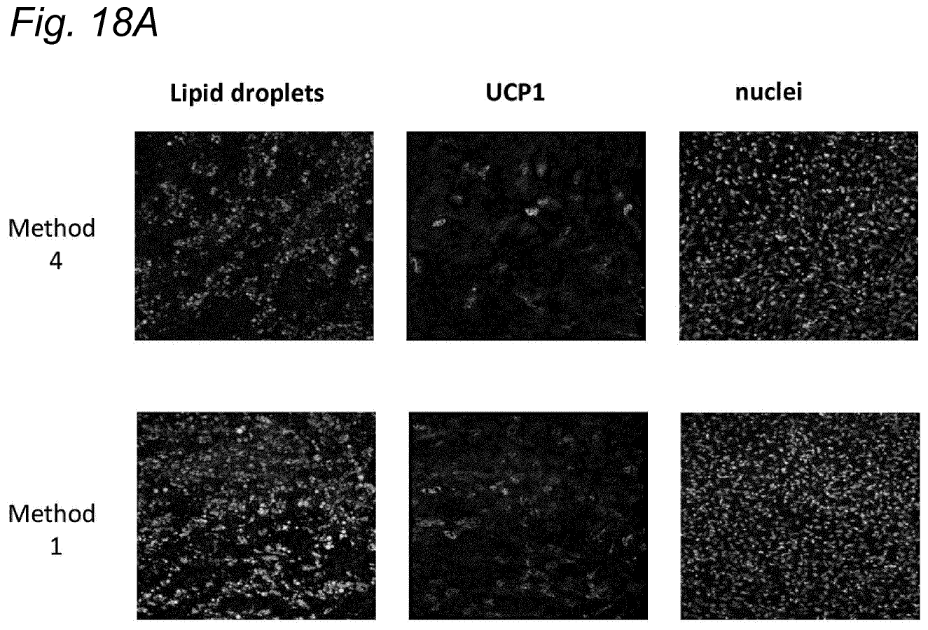

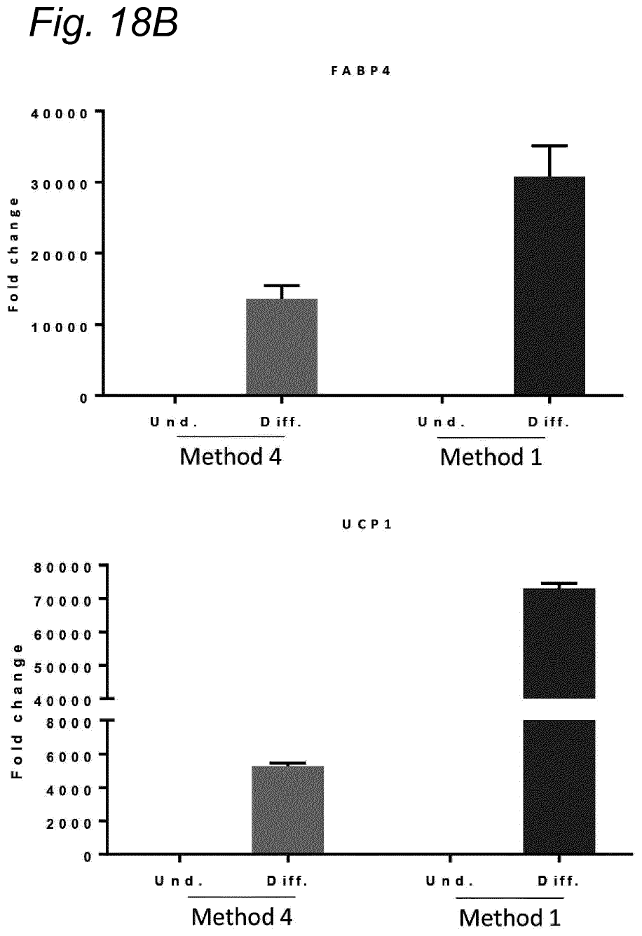

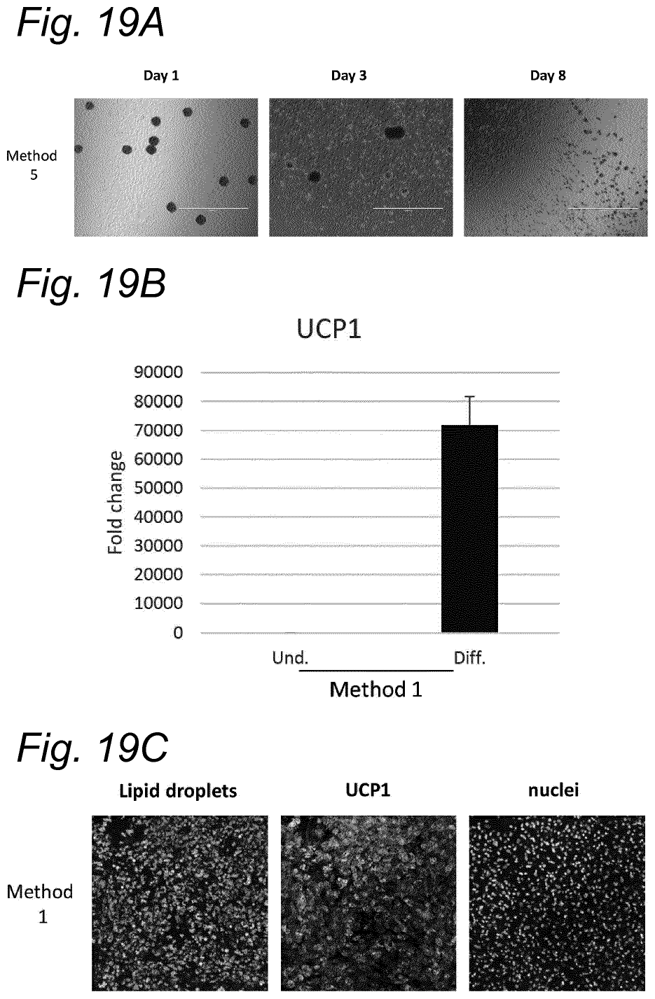

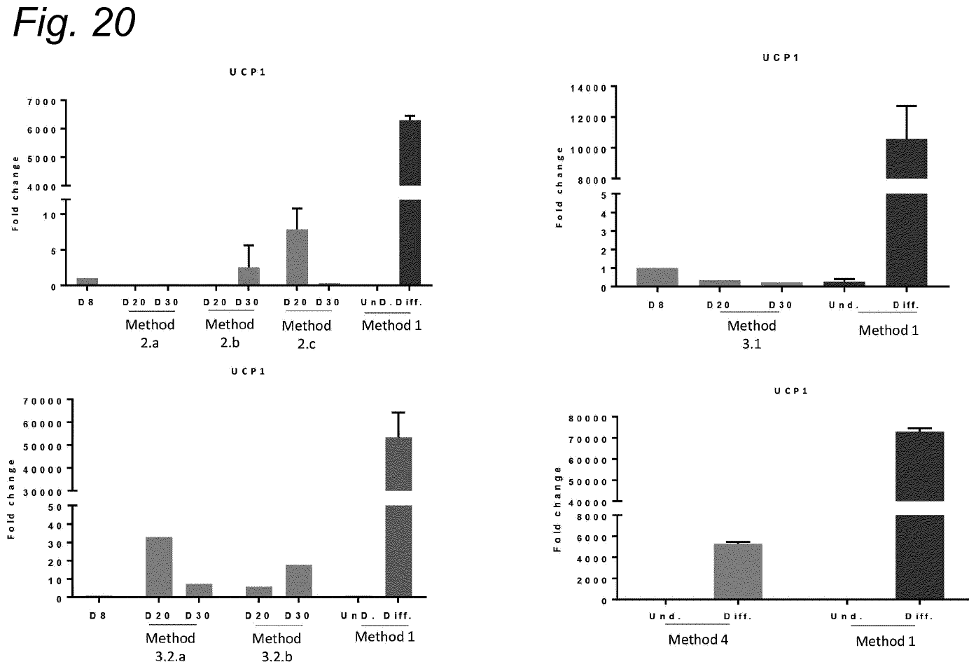

Importantly, to complete the characterization, BAP derived at day 12 or 16 and after 5 passages were differentiated for 17 days and then treated with forskolin (10 μM). After 24 hours, we observed an increase of the free glycerol released (by about 60%) for the treated cells compared to untreated cells (FIG. 13.D). The BA were able able to respond to forskolin to increase the lipolysis, thus showing the functionality of these cells.

These results show without doubt that the method of the invention generates not only adipocytes, but specifically brown adipose progenitors and adipocytes which are functional, with very high yield (up to 85% purity) that is unprecedented (see Example 2), allowing to obtain a population of BA with a high purity amenable to industrial applications.

Time Interval to Derive BAP

As described above, before the enrichment of BAP with the consecutive passages, the cells are cultivated in myogenic medium from day 6 to day 22 and then maintained in medium containing serum.

The BAP derived at different time points were differentiated (as described in example 1) for 17 days (data shown for passage 5). The BA cultures all expressed strongly the UCP1 protein with numerous lipid droplets (FIG. 13, A). The quantification of the number of cells expressing UCP1 and presenting lipid droplets showed a range of 43% (for BAP derived at day 14 to 69% (for BAP derived at day 12). The expression of UCP1 is confirmed by qPCR analysis (FIG. 13, B). Both FABP4 and UCP1 were, as expected, more expressed in BA than in BAP. These data show that BAP can be derived at any timepoint from day 12 to day 22 and lead to the generation of BA with a satisfying efficiency. Furthermore, after stimulation with forskolin, we observed an increase of the lipolysis for BA obtained after differentiation of BAP derived at day 12 and 16, confirming previous results (FIG. 13.D).

In FIG. 14, the method of the invention was performed without step c) in order to assess the importance of this step. The BAP were derived at day 20 with (1) or without (2) a culturing step in medium containing serum. After differentiation, the rate of UCP1-positive cells is 60% for the BAP derived with this additional step and 23% for those derived without it. These results show that while step c) significantly increases the yield of BA generation, its absence does not prevent the obtention of a BA population, and is therefore optional.

These results show that after induction of the iPAM cells, BAP and BA can be generated with high yield whatever the timepoint selected for BAP derivation, as long as this timepoint is comprised between 12 and 22 days of primary differentiation, followed by 2 to 9 serial passages. Among these possibilities, the preferred conditions are to derive BAP at day 16 and passage them 5 times before differentiation.

### Example 2

In this example, the method of the invention was compared to other methods identified in the prior art as claiming to generate BAP and/or BA, in order to ascertain the superiority of the method described in the present application. For each method (2 to 5), BAP and BA were also generated in parallel using the method of the invention as described below (method 1), using the same batch of undifferentiated hiPS cells at the same time.

### Methods

The full names and manufacturers of all compounds used herein are detailed in the Appendix

Method 1: Primary Differentiation and Derivation of hBAP From hiPS Cells and Differentiation of hBAP Into hBA:

Step a) Undifferentiated hiPS cells are dissociated to single cells using trypsin and seeded at a density of 5.5.10⁴ cells/cm² on matrigel-coated dishes in mTESR-1 medium supplemented with Rock-1 inhibitor (10 μM). One day after, medium is changed to fresh mTESR-1 without Rock-1 inhibitor. When the cells form small aggregates, determined at the day 0 of primary differentiation, they are changed to a sequence of differentiation media.

At day 0, medium is changed to medium composed of DMEM supplemented with ITS (1%), CHIR99021 (3 μM) and LDN-193189 (500 nM). The medium is refreshed daily until day 6.

Step b) At day 6, medium is changed to medium composed of DMEM supplemented with KSR (15%), LDN-193189 (500 nM), HGF (10 ng/ml) and IGF-1 (2 ng/ml). The medium is changed daily until day 8.

At day 8, medium is changed to medium composed of DMEM supplemented with KSR (15%) and IGF-1 (2 ng/ml). The medium is changed daily until day 12.

From day 12 to day 16, medium is changed to medium composed of DMEM supplemented with KSR (15%), HGF (10 ng/ml) and IGF-1 (2 ng/ml). The medium is changed every 2 days (FIGS. 6 and 7).

Step c) At day 16, medium is changed to a medium composed of DMEM supplemented with FBS (10%). The medium is refreshed every 2 days.

Step d) After 7 days, the cells are passaged using trypsin and seeded on tissue culture grade plate. (passage number 0). Cells are maintained in the previous medium composed of DMEM supplemented with FBS (10%) and FGF-2 (5 ng/ml).

When the cells reach confluence, they are passaged and seeded at a density of 5.10⁴ cells/cm².

Passages are repeated 5 times until a cell population with homogeneous morphology is obtained .

Step e) The hBAP are plated at a density of 5.10⁴ cells/cm² and maintained in derivation medium i.e. DMEM supplemented with FBS (10%) and FGF-2 (5 nh/ml). When the cells reach confluence, determined at the day 0 of secondary differentiation, the medium is changed to differentiation medium composed of DMEM with low glucose (1 g/l) supplemented with FBS (10%), rosiglitazone (1 μM), insulin (10 μg/ml), T3 (0.2 nM), SB431542 (5 μM), ascorbic acid (25.5 μg/ml), EGF (10 ng/ml), hydrocortisone (4 μg/ml), dexamethasone (1 μM) and IBMX (500 μM). Dexamethasone and IBMX are discarded after day 3. The differentiation medium is then changed twice a week until day 17 of the secondary differentiation.

Method 2: Protocol Described in WO2013/030243

WO2013/030243 claims a method for preparing populations comprising adipocytes by culturing a population of iPAM cells under appropriate conditions for their differentiation into adipocytes, i.e. in the presence of an efficient amount of at least one or more compounds known to induce adipocyte differentiation. In order to determine whether BAP and BA comparable to method 1 can be obtained using the method of WO2013/030243, iPAM cells were generated and subsequently exposed to either a myogenic culture medium (2.a.) or an adipogenic medium (2.b. and 2.c.), adipogenic media being considered potential “appropriate conditions” for the generation of adipocytes.

Generation of iPAM Cells

Undifferentiated hiPS cells are dissociated to single cells using trypsin and seeded on matrigel-coated dishes in mTESR-1 medium supplemented with Rock-1 inhibitor (10 μM). One day after, medium is changed to fresh mTESR-1 without Rock-1 inhibitor. When the cells form small aggregates, determined at the day 0 of primary differentiation, they are changed to a sequence of differentiation media.

At day 0, medium is changed to medium composed of DMEM supplemented with ITS (1%), CHIR99021 (3 μM) and LDN-193189 (500 nM).

After 3 days, the previous medium is supplemented with FGF-2 (20ng/ml). The medium is refreshed daily until day 6.

Method 2.a: Generation of iPAM Cells and Culturing Step in Myogenic Medium:

At day 6, medium is changed to medium composed of DMEM supplemented with KSR (15%), LDN-193189 (500 nM), HGF (10 ng/ml), FGF-2 (20 ng/ml) and IGF-1 (2 ng/ml). The medium is changed daily until day 8.

At day 8, the medium is changed to medium composed of DMEM supplemented with KSR (15%) and IGF-1 (2 ng/ml).

At day 12, the medium is changed to medium composed of DMEM supplemented with KSR (15%), HGF (10ng/ml) and IGF-1 (2 ng/ml). Medium is refreshed every 2-3 days.

Method 2. b and c: Generation of iPAM Cells and Culturing Step in Adipogenic Medium

iPAM cells are generated as described above.

At day 6, medium is changed to a medium composed of DMEM supplemented with KSR (15%), LDN-193189 (500 nM), HGF (10 ng/ml), FGF-2 (20 ng/ml) and IGF-1 (2 ng/ml). The medium is changed daily until day 8. At day 8, medium is then changed to one of two adipocyte differentiation media:

b. An adipocyte differentiation medium composed of DMEM-based medium containing 15% KSR and supplemented with dexamethasone (1 μM), IBMX (500 μM), insulin (10 μg/ml), T3 (0,2 nM) and rosiglitazone (1 μM). This medium would be considered a standard adipogenic medium by a person skilled in the art.

c. The adipocyte differentiation medium described in step e) of the present application. The medium is composed of DMEM-based medium containing 15% KSR, dexamethasone (1 μM), IBMX (500 μM), insulin (10 μg/ml), T3 (0,2 nM), SB431542 (5 μM), ascorbic acid (25,5 μg/ml), EGF (10 ng/ml) and hydrocortisone (4 μg/ml).

At day 11, dexamethasone and IBMX are removed for both adipogenic media. The medium is refreshed every 2-3 days.

Method 3: Protocol Described in WO17223457

WO17223457 claims an in vitro method of generating induced Brown Adipose Tissue (iBAT) cells that express UCP1 by providing a population of iPAM cells followed by culturing this population under 2 different sets of conditions: “HIFL” or “PRA-Adipomix”. In order to determine whether BAP and BA comparable to method 1 can be obtained using method 3, iPAM cells were generated and subsequently exposed each of the methods described in WO17223457.

Method 3.1. Method «HIFL»:

Day 0 to day 6: see previously described in Method 2, «Generation of iPAM cells»

At day 6, medium is changed to a medium composed of DMEM supplemented with KSR (15%), LDN-193189 (100 nM), HGF (10 ng/ml), FGF-2 (20 ng/ml) and IGF-1 (2 ng/ml). The medium is changed daily until day 8.

At day 8, the medium is only supplemented with HGF (10 ng/ml) and IGF-I (2 ng/ml). The medium is changed every 2-3 days.

Method 3.2. Method «PRA-Adipomix»:

Day 0 to day 6: see previously described in Method 2, «Generation of iPAM cells»

At day 6, medium is changed to DMEM-based medium containing PD173074 (250 nM and retinoic acid (100 nM).

At day 8, cultures are changed to two adipocyte differentiation media:

a. The Adipomix medium described into WO17223457. The medium is composed of DMEM based medium containing 15% KSR, 1X insulin-transferrin-selenium (ITS), 500 μM IBMX, 125 nM indomethacin, 1 nM T3, 5 μM dexamethasone and 1 μM rosiglitazone. The medium is refreshed every 2-3 days.

b. The adipocyte differentiation medium described in the present application. The medium is composed of DMEM based medium containing 15% KSR, dexamethasone (1 μM), IBMX (500 μM), insulin (10 μg/ml), T3 (0,2 nM), rosiglitazone (1 μM), SB431542 (5 μM), ascorbic acid (25,5 μg/ml), EGF (10 ng/ml) and hydrocortisone (4 μg/ml). At day 11, dexamethasone and IBMX are removed. The medium is refreshed every 2-3 days.

Method 4: Protocol Described in «Hafner et al., 2016»

Hafner et al. discloses a method to generate BAP and BA cells from hiPS cells by forming embryoid bodies (EBs), culturing them in a medium containing DMEM, serum and FGF-2, followed by serial passaging and culture in the adipogenic medium of step e). Methods 1 and 4 share common steps to derive the BAP and differentiate them into BA, but the differ by the first steps of differentiation of the pluripotent cells with the formation of embryoid bodies for method 4 or the generation of iPAM cells followed by exposure to a myogenic medium for method 1.

In order to determine whether BAP and BA can be obtained using method 4 with a yield comparable to that of method 1, both methods were conducted in parallel.

EBs were formed by floating culture in DMEM/F12 medium supplemented with 20% Knock-out Serum Replacement. Ten days after EBs formation, EBs are plated on gelatin-coated culture plates and maintained in DMEM/F12 medium supplemented with 20% KSR for 8 days. At day 18, medium is changed to a medium composed of DMEM supplemented with 10% FBS. After a week, the cells are passaged and seeded on tissue culture grade plates. Passages are repeated until a cell population with homogeneous morphology is obtained, after passage 4 or 5.

The cells are plated at a density of 5.10⁴ cells/cm² and maintained in derivation medium i.e. DMEM supplemented with FBS (10%) and FGF-2 (5 nh/ml). When the cells reach confluence, determined at the day 0 of differentiation, the medium is changed to differentiation medium composed of DMEM with low glucose (1 g/l) supplemented with FBS (10%), rosiglitazone (1 μM), insulin (10 μg/ml), T3 (0.2 nM), SB431542 (5 μM), ascorbic acid (25.5 μg/ml), EGF (10 ng/ml), hydrocortisone (4 μg/ml), dexamethasone (1 μM) and IBMX (500 μM). Dexamethasone and IBMX are discarded after day 3. The differentiation medium is then changed twice a week until day 17 of the differentiation.

Method 5: Protocol Described in WO2012/147853

WO2012/147853 claims a method for the high-efficiency (>90%) production of brown adipocytes from hiPS cells with a 2 step-process: first, cells aggregates are produced by floating culture from pluripotent stem cells in the presence of a hematopoietic cytokine in a serum-free environment. Then, BA are generated by cell adhesion of the cell aggregates in the presence of a hematopoietic cytokine. Methods 1 and 5 were conducted in parallel in order to compare the yield of differentiation of BA obtainable by each method.

According to WO2012/147853, the differentiation of hiPS cells is initiated by the formation of embryoid bodies (EBs) by floating culture in IMDM/F12 medium (containing 5 mg/mL BSA, 1% by volume synthetic lipid solution, 1% by volume of 100× ITS, 450 mM MTG, 2 mM L-glutamine, 5% by volume of PFHII, 50 mg/mL of ascorbic acid, 20 ng/mL of BMP4, 5 ng/mL of VEGF, 20 ng/mL of SCF, 2.5 ng/mL of Flt3L, 2.5 ng/mL of IL6, and 5 ng/mL of IGF2). Medium is changed every 3 days.

After 8 days, EBs are plated on gelatin-coated culture plates in IMDM/F12 medium (containing 5 mg/mL BSA, 1% by volume of a synthetic lipid solution, 1% by volume of 100× ITS, 450 mM MTG, 2 mM L-glutamine, 5% by volume of PFHII, 50 mg/mL of ascorbic acid, 10 ng/mL of BMP7, 5 ng/mL of VEGF, 20 ng/mL of SCF, 2.5 ng/mL of Flt3L, 2.5 ng/mL of IL6, and 5 ng/mL of IGF2) for one week. Medium is changed every 3 days.

Quantitative RT-PCR:

Total RNA was extracted from cell cultures using the nucleo spin RNA plus kit (Macherey-Nagel). RT-PCR was performed on 500 ng total RNA using iScript gDNA clear cDNA synthesis Kit (Biorad), appropriate primers and run on a LightCycler 48011 (Roche). TBP was used as the internal control.

Immunocytochemistry:

Cell cultures were fixed with PFA 4%. Cells were incubated for 30 minutes with a blocking solution composed of 5%NGS, 1% fetal bovine serum and 0.2%Triton in Phosphate Buffered Saline (PBS). Primary antibodies incubation was performed during 1h30 at room temperature and antibodies working dilutions were as follow: anti-UCP1 (R&D) was 1:250, anti-Desmin (Santa Cruz) was 1:800. After PBS washing, cells were incubated with AlexaFluor488-conjugated secondary antibodies (Invitrogen) at 1:1000 for 30 minutes, and counterstained with Dapi. HCS lipidtox neutral lipid staining was done according to standard protocol.

Experimental Results

Comparison of the Method 1 to the Method 2:

The ability of iPAMs cells to generate brown adipocytes (FIG. 15) was evaluated, with and without applying the culturing steps described in the method of the present invention. Starting from a single batch of hiPS cells at the same time, iPAM cells were generated. Then, the cells were maintained in myogenic medium (method 2.a),cultivated in adipogenic medium (methods 2.b and c), or differentiated according to method 1.

Expression of adipocyte markers analysed by qPCR: For this comparison, the expression of the genes at day 20, day 30 and for the BAP and BA of the method 1) were normalized against a sample taken at day 8 (i.e. before any commitment to adipocyte lineage). After 20 or 30 days of differentiation the cells of method 2.a. weakly expressed FABP4 (pan-adipogenic marker) but didn't express UCP1 (brown adipocyte specific marker). Expression of UCP1 was however detected at low levels in cells of methods 2.b. and 2.c. at day 20 or 30. Expression of UCP1 was 900 times higher in the control cells generated from method 1.

These data show that with appropriate induction using adipogenic media, iPAM cells can differentiate into brown adipocytes. However, the expression of UCP1 was significantly lower in cells generated with methods 2.b. or 2.c than with in cells produced with method 1, demonstrating the importance of the additional steps described in the present invention. The combination of culturing steps constituting the present invention generate a surprisingly high yield of BA production compared to any other combination of those steps as would be suggested in prior art.

Comparison of the Method 1 to the Method 3:

The ability to generate BA cells according to WO17223457 was evaluated. Starting from a single batch of hiPS cells at the same time, iPAM cells were generated. Then, the cells were exposed to the culturing steps of methods 1, 3.1., 3.2.a. and 3.2.b. (detailed in the section methods).

Expression of Adipocyte Markers Analysed by qPCR:

For this comparison, the expression of the genes at day 20, day 30 and for the BAP and BA of the method 1) were normalized against a sample taken at day 8 (i.e. before any commitment to adipocyte lineage).

After 20 or 30 days of differentiation the cells of method 3.1. named “HIFL” didn't express any adipocyte markers (FIG. 16) suggesting that the cells did not differentiate into brown adipocytes. After 20 or 30 days, cells of methods 3.2. expressed low levels of FABP4 and UCP1 markers (FIG. 17). Expression of UCP1 was at least 1000 times higher in the control cells generated from method 1.

These data again show that with appropriate induction, iPAM cells can differentiate into brown adipocytes. However, the expression of UCP1 was significantly lower in cells generated with methods 3 compared to method 1. While the methods described in WO17223457 may generate BA cells with low yield, these results clearly demonstrate the superiority of the method of the present invention.

Comparison of the Method 1 to the Method 4:

The ability to generate BA cells according to Hafner et al. was evaluated. hiPS cells were differentiated to generate brown adipocytes according to the method 1 or the method 4. BAP cultures before secondary differentiation and BA cultures after 17 days of maturation in adipogenic medium were analysed by qPCR and immunofluorescence at passage 4 (data not shown) and 5 (FIG. 18).

Expression of adipocyte markers analysed by qPCR: For this comparison, the expression of the genes in the differentiated cells are normalized by the expression in the undifferentiated cells. BA cells obtained with both methods showed expression of FABP4 and UCP1 (FIG. 18.B.), but cells from method 1 expressed UCP1 a levels 100 times higher.

Expression of adipocyte markers analysed by IF: The population of cells expressing UCP1 and presenting lipid droplets was significantly higher with the method 1 than with the method 4 (about 47% for the method 1 and 25% for the method 4) (FIG. 18.A.)

Thus, these results teach that surprisingly, the combination of the induction of paraxial mesoderm lineage and the enrichment of BAP by passaging as described in the method 1 significantly improves the yield of differentiation.

Comparison of the Method 1 to the Method 5:

We could not reproduce the results of method 5. Despite several attempts, after the first step of the formation of the cell aggregates, a high cellular mortality was observed after 2 or 3 days (FIG. 19). Cell cultures were completely lost after 8 days.

Control cells differentiated according to method 1 using the same batch of undifferentiated hiPS cells however showed expected results whether by qPCR (FABP4 and UCP1 markers were more expressed in BA than in BAP) or by immunofluorescence (after 17 days of differentiation, the cell population expressing UCP1 and presenting lipid droplets reached 85%), suggesting that the cellular mortality observed during the reproduction of the method 5 was not due to the undifferentiated hiPS cells used.

These results suggest that the method of WO2012/147853 is either not reproducible or highly dependent on external factors, which make this method unsuitable for industrial applications, ascertain the superiority of method 1 and suggest that the cells which can theoretically be obtained from method 5 are not comparable to the cells obtained by the method of the present invention.

Summary of the Comparisons:

To demonstrate the technical advantages of the present invention over existing protocols, we have compared it to other protocols disclosed in the prior art which either claim the production of:
adipocytes or brown adipocytes from iPAM cells, or
brown adipocytes from hiPS cells.

The analysis by qPCR (FIG. 20) showed that the BA generated with method 1 expressed the brown adipocyte specific marker UCP1 at higher level than any of the other protocols (e.g. from 100 times more than method 4 and up to 1000 times more than method 3) testifying to a significantly higher yield of differentiation. These results clearly demonstrate that the method of the present invention is superior to generate BAP and BA compared to existing protocols in terms of yield and/or duration but also of robustness with the absence of EBs. The present application therefore discloses a surprising novel way to generate BAP and BA.

### REFERENCES

Throughout this application, various references describe the state of the art to which this invention pertains. The disclosures of these references are hereby incorporated by reference into the present disclosure.
Altschul, S. F. et al., 1990, “Basic local alignment search tool”, J. Mol. Biol. 215:403-410.
Beggs et al. 1992 J Biological chemistry. PMID:1339456.
Biocomputing: Informatics and Genome Projects, Smith, D. W., ed., Academic Press, New York, 1993;
BLAST Manual, Altschul, S., et al., NCBI NLM NIH Bethesda, Md. 20894.
Buckingham et al., Dev Cell., 2014. DOI: http://dx.doi.org/10.1016/j.devce1.2013.12.020
Cannon and Nedergaard. Brown adipose tissue: function and physiological significance. 2004, Physiol Rev., 84, 277-35910.1152/physrev.00015.2003.
Carillo, H., and Lipman, D., SIAM J. Applied Math., 48: 973-1073 (1988).
Chal J. et al., (2015), Nature Biotechnology, Differentiation of pluripotent stern cells to muscle fiber to model Duchenne muscular dystrophy 2015, doi :10.1038/nbt.3297.
Clevers, H. (2006). Wnt/beta-catenin signaling in development and disease. Cell 127, 469-80.
Cohen, P. and Goedert, M. (2004). GSK3 inhibitors: development and therapeutic potential. Nat Rev Drug Discov 3, 479-87.
Computational Molecular Biology, Lesk, A. Miller., ed., Oxford University Press, New York, 1988;
Computer Analysis of Sequence Data, Part I, Griffin, A. M., and Griffin, H. G., eds., Humana Press, New Jersey, 1994;
Cuny G D, Yu P B, Laha J K, Xing X, Liu J F, Lai C S, Deng D Y, Sachidanandan C, Bloch KD, Peterson R T. Structure-activity relationship study of bone morphogenetic protein (BMP) signaling inhibitors. Bioorg Med Chem Lett. 2008 Aug. 1; 18(15):4388-92. Epub 2008 Jun. 27.
Devereux, J., et al., 1984, “A comprehensive set of sequence analysis programs for the VAX”, Nucleic Acids Research 12 (1): 387-395.
Gazerro E. and Minetti C. (2007) Potential drug targets within bone morphogenetic protein signaling pathways. Curr Opin Pharmacol.: 325-33
Huang et al., Scientific reports, 2017. doi:10.1038/srep40716
Loser, P., Schirm, J., Guhr, A., Wobus, A. M. and Kurtz, A. (2010). Human embryonic stem cell lines and their use in international research. Stem Cells 28, 240-6.
Meyers E. X. and Miller W., Optimal alignments in linear space, (1988) Comput. Appl. Biosci., 4:11-17.
Montcouquiol, M., Crenshaw, E. B., 3rd and Kelley, M. W. (2006). Noncanonical Wnt signaling and neural polarity. Annu Rev Neurosci 29, 363-86.
Needleman and Wunsch, 1970, “A general method applicable to the search for similarities in the amino acid sequence of two proteins”, J. Mol. Biol. 48:443-453.
Nedergaard et al 2001, Biochim Biophys Acta. UCP1: the only protein able to mediate adaptive non-shivering thermogenesis and metabolic inefficiency
Paulin et al., Exp cell research, 2004. DOI:10.1016/j.yexcr.2004.08.004
Sato, N., Meijer, L., Skaltsounis, L., Greengard, P. and Brivanlou, A. H. (2004). Maintenance of pluripotency in human and mouse embryonic stem cells through activation of Wnt signaling by a pharmacological GSK-3-specific inhibitor. Nat Med 10, 55-63.
Schlessinger, K., Hall, A. and Tolwinski, N. (2009). Wnt signaling pathways meet Rho GTPases. Genes Dev 23, 265-77.
Sequence Analysis in Molecular Biology, von Heine, G., Academic Press, 1987
Sequence Analysis Primer, Gribskov, M. and Devereux, J., eds., M Stockton Press, New York, 1991
Taelman, V. F., Dobrowolski, R., Plouhinec, J. L., Fuentealba, L. C., Vorwald, P. P., Gumper, I., Sabatini, D. D. and De Robertis, E. M. (2010). Wnt signaling requires sequestration of glycogen synthase kinase 3 inside multivesicular endosomes. Cell 143, 1136-48.
Takahashi, K., Tanabe, K., Ohnuki, M., Narita, M., Ichisaka, T., Tomoda, K. and Yamanaka, S. (2007). Induction of pluripotent stem cells from adult human fibroblasts by defined factors. Cell 131, 861-72.
Takahashi, K. and Yamanaka, S. (2006). Induction of pluripotent stem cells from mouse embryonic and adult fibroblast cultures by defined factors. Cell 126, 663-76.
Wu, D. and Pan, W. (2010). GSK3: a multifaceted kinase in Wnt signaling. Trends Biochem Sci 35, 161-8.
Yoon J K et al, (2015), The bHLH regulator pMesogeninl is required for maturation and segmentation of paraxial mesoderm. Genes. Dev., 14: 3204-3214.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R. et al. (2007). Induced pluripotent stem cell lines derived from human somatic cells. Science 318, 1917-20.
Yu P B, Hong C C, Sachidanandan C, Babitt J L, Deng D Y, Hoyng S A, Lin H Y, Bloch K D, Peterson R T. Dorsomorphin inhibits BMP signals required for embryogenesis and iron metabolism. Nat Chem Biol. 2008 January; 4(1):33-41. Epub 2007 Nov. 18.
Zammit et al., J. Cell Science, 2006. doi: 10.1242/jcs.02908

### Abbreviations/Molecules List

Ascorbic acid: (also known as vitamin C) is an essential nutrient in human diets. Ascorbic acid is a potent reducing and antioxidant agent. Besides anti-oxidant activity, ascorbic acid (ASC) acts as a cofactor of the hydroxylating enzyme of proline and lysine residues in procollagen. Manufacturer: Sigma Aldrich
CHIR99021: is an aminopyrimidine derivative that is an extremely potent inhibitor of GSK3 and functions as a WNT activator. Manufacturer: axon medchem
Dexamethasone: is a synthetic glucocorticoid hormone. Manufacturer: Sigma Aldrich
DMEM: Dulbecco's Modified Eagle Medium (basal culture medium). Manufacturer: Gibco
EGF: epidermal growth factor (EGF) stimulates cell growth and differentiation by binding to its receptor, EGFR. Manufacturer: Miltenyi biotech
FBS: Fcetal bovine serum . Manufacturer: PanSera; Dutscher
FGF-2 (also called bFGF): fibroblast growth factor-2. Manufacturer: Miltenyi biotech
HGF: hepatocyte growth factor. Manufacturer: R&D systems
Hydrocortisone: is a glucocorticoid secreted by the adrenal cortex. Manufacturer: Sigma aldrich
IBMX: (3-isobutyl-1-methylxanthine) is non-specific inhibitor of cyclic AMP and cyclic GMP phosphodiesterases (PDEs). By inhibiting PDEs, IBMX increases cellular cAMP and cGMP levels, activating cyclic-nucleotide-regulated protein kinases. Manufacturer: Stemcell technologies
IGF-1: Insulin growth factor type 1. Manufacturer: Miltenyi biotech
ITS: Insulin-Transferrin-Selenium. It's a cell supplement. Insulin promotes glucose and amino acid absorption, lipogenesis, intracellular transport, and protein and nucleic acid synthesis. Transferrin is a iron-binding glycoprotein that controls the level of free-iron (can also help to reduce the level of oxygen and peroxide free radicals). Selenium is a cofactor for glutathione peroxidase and other proteins, and is used as an antioxydant in culture media. Manufacturer: Gibco
KSR: Knockout serum replacement. It's a more defined, FBS-free medium supplement that supports the growth of pluripotent stem cells (PSCs). Manufacturer: Gibco
LDN-193489: LDN-193189 is a cell permeable small molecule inhibitor of bone morphogenetic protein (BMP) type I receptors ALK2 and ALK3. LDN-193189 was derived from structure-activity relationship studies of Dorsomorphin and functions primarily through prevention of Smad1, Smad5, and Smad8 phosphorylation. Manufacturer: Miltenyi biotech
mTESR-1: Standardized Medium for the Feeder-Independent Maintenance of hESCs & hiPSCs. Manufacturer: Stemcell technologies
Y-27632 (usually called: Rock-1 inhibitor): inhibitor of Rho-associated, coiled-coil containing protein kinase (ROCK). Manufacturer: Tocris bioscience.
Rosiglitazone: Rosiglitazone is an anti-diabetic drug from the thiazolidinedione class. Like other thiazolidinediones, its mechanism of action is by activation of the intracellular receptor class of the peroxisome proliferator-activated receptors (PPARs), specifically PPAR-gamma. Manufacturer: Prestwick
SB431542: is a small molecule inhibitor of the TGF-6/Activin/NODAL pathway that inhibits ALK5, ALK4, and ALK7, but does not inhibit the BMP type I receptors ALK2, ALK3, and ALK6. Manufacturer: Stemcell technologies.
T3: triiodothyronine. T3 is a thyroid hormone resulting of deiodination of thyroxine. Manufacturer: Sigma aldrich.

1. A method for preparing Brown Adipocyte Progenitor (BAP) or Brown Adipocyte (BA) cells, said method comprising the following steps:
a) Culturing pluripotent cells in a culture medium comprising an activator of the Wnt signaling pathway to obtain induced paraxial mesoderm progenitor (iPAM) cells;
b) Culturing said iPAM cells in a myogenic culture medium
c) Optionally further culturing cells obtained at the end of step b) in a culture medium with serum or an equivalent thereof, optionally further comprising FGF2 or an equivalent thereof;
d) Selecting BAP cells by passaging the cells obtained at the end of step b) or c) and seeding them into culture dish; and
e) Optionally culturing selected BAP cells preferably those obtainable at the end of step d) in an adipogenic culture medium comprising serum or an equivalent thereof obatining BA cells.

2. (canceled)

3. The method according to claim 1, wherein step a) is carried out in a culture medium further comprising an inhibitor of the Bone Morphogenetic Pathway (BMP) signaling pathway and optionally DMSO.

4. The method according to claim 1, wherein:
a) the Wnt signaling pathway is the canonical Wnt/beta catenin signaling pathway and/or the Wnt/PCP signaling pathway,
b) the inhibitor or the BMP signaling pathway is selected from the group consisting of: Noggin, Chordin, Chordin-like 1-3, Follistatin, Follistatin-like 1-5, a member of the Dan family and variants and fragments thereof.

5. The method, according to claim 1, wherein step b) is carried out using a myogenic culture medium that comprises or consists of or essentially consists of a culture medium, serum or an equivalent thereof, an inhibitor of a BMP receptor, an activator of the c-MET receptor and an activator of an IGF or insulin receptor

6. The method according to claim 1, wherein step e) is carried out using an adipogenic culture medium that comprises or essentially consists of a culture medium, an inhibitor of the TGFbeta/Activin/NODAL pathway (preferably SB431542), an activator of the EGF (Epidermal Growth Factor) receptor (preferably EGF), ascorbic acid, and an activator of a corticoid receptor (preferably hydrocortisone).

7. The method according to claim 1, wherein BA cells are characterized by the expression of UCP1

8. The method according to claim 1, wherein BAP cells are characterized by their ability to be converted into BA cells expressing UCP1.

9. A population of BA or BAP cells obtainable by the method of claim 1, wherein the population of BA cells comprises at least 10%, 15%, 20%, 25%, 30%, 35%, 40%, 45%, 50%, 55%, 60% 65%, 70%, 75%, 80%, 85%, 90%, 95%, 99% or 100% of cells expressing UCP1 or wherein the population of BAP cells is characterized by the ability to be converted into a population of BA cells comprising at least 10%, 15%, 20%, 25%, 30%, 35%, 40%, 45%, 50%, 55%, 60%, 65%, 70%, 75%, 80%, 85%, 90%, 95%, 99% or 100% of cells espression UCP1.

10.-12. (canceled)

13. A method for treating a disease or condition linked with BA or BAP cell activity and preferably being a metabolic disease or condition such as obesity-related pathologies, metabolic syndrome, diabetes mellitus, hyperlipidemia, NASH (Non-Alcoholic Steato Hepatitis), Energy balance (intake versus expenditure), comprising administering a population of BA or BAP cells defined in cliam 9.

14. (canceled)


SEQUENCE LISTING (13 sequences)

SEQ ID NO: 1 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 2 - protein, 277 aa, Mus musculus
MHLRLISCFFIILNFMEYIGSQNASRGRRQRRMHPNVSQGCQGGCATCSDYNGCLSCKPRLFFVLERIGMKQIGVCLSSCPSGYYGTRYPDINKCTKCKVDCDTCFNKNFCTKCKSGFYLHLGKCLDSCPEGLEANNHTMECVSIVHCEASEWSPWSPCMKKGKTCGFKRGTETRVRDILQHPSAKGNLCPPTSETRTCIVQRKKCSKGERGKKGRERKRKKLNKEERKETSSSSDSKGLESSIETPDQQENKERQQQQKRRARDKQQKSVSVSTVH

SEQ ID NO: 3 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 4 - protein, 243 aa, Mus musculus
MRFCLFSFALIILNCMDYSQCQGNRWRRNKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLDETMECVEGCEVGHWSEWGTCSRNNRTCGFKWGLETRTRQIVKKPAKDTIPCPTIAESRRCKMAMRHCPGGKRTPKAKEKRNKKKRRKLIERAQEQHSVFLATDRVNQ

SEQ ID NO: 5 - protein, 292 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSGIEVTLAEGLTSVSQRTQPTPCRRRYL

SEQ ID NO: 6 - protein, 176 aa, Homo sapiens
MRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 7 - protein, 177 aa, Homo sapiens
FRLFSFALIILNCMDYSHCQGNRWRRSKRGCRIENCDSCFSKDFCTKCKVGFYLHRGRCFDECPDGFAPLEETMECVGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 8 - DNA, 567 bp, Mus musculus
atggacaacctgggtgagaccttcctcagcctggaggatggcctggactcttctgacaccgctggtctgctggcctcctgggactggaaaagcagagccaggcccttggagctggtccaggagtcccccactcaaagcctctccccagctccttctctggagtcctactctgaggtcgcactgccctgcgggcacagtggggccagcacaggaggcagcgatggctacggcagtcacgaggctgccggcttagtcgagctggattacagcatgttggcttttcaacctccctatctacacactgctggtggcctcaaaggccagaaaggcagcaaagtcaagatgtctgtccagcggagacggaaggccagcgagagagagaaactcaggatgcggaccttagccgatgccctccacacgctccggaattacctgccgcctgtctacagccagagaggccaaccgctcaccaagatccagacactcaagtacaccatcaagtacatcggggaactcacagacctcctcaacagcagcgggagagagcccaggccacagagtgtgtga

SEQ ID NO: 9 - DNA, 582 bp, Homo sapiens
atggacaacctgcgcgagactttcctcagcctcgaggatggcttgggctcctctgacagccctggcctgctgtcttcctgggactggaaggacagggcagggccctttgagctgaatcaggcctccccctctcagagcctttccccggctccatcgctggaatcctattcttcttctccctgtccagctgtggctgggctgccctgtgagcacggcggggccagcagtgggggcagcgaaggctgcagtgtcggtggggccagtggcctggtagaggtggactacaatatgttagctttccagcccacccaccttcagggcggtggtggccccaaggcccagaagggcaccaaagtcaggatgtctgtccagcggaggcggaaagccagcgagagggagaagctcaggatgaggaccttggcagatgccctgcacaccctccggaattacctgccacctgtctacagccagagaggccagcctctcaccaagatccagacactcaagtacaccatcaagtacatcggggaactcacagacctccttaaccgcggcagagagcccagagcccagagcgcgtga

SEQ ID NO: 10 - protein, 232 aa, Mus musculus
MERCPSLGVTLYALVVVLGLRAAPAGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 11 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 12 - protein, 53 aa, Homo Sapiens
NSDSECPLSHDGYCLHDGVCMYIEALDKYACNCVVGYIGERCQYRDLKWWELR

SEQ ID NO: 13 - protein, 307 aa, Homo Sapiens
MGGLTASDVHPTLGVQLFSAGIAACLADVITFPLDTAKVRLQVQGECPTSSVIRYKGVLGTITAVVKTEGRMKLYSGLPAGLQRQISSASLRIGLYDTVQEFLTAGKETAPSLGSKILAGLTTGGVAVFIGQPTEVVKVRLQAQSHLHGIKPRYTGTYNAYRIIATTEGLTGLWKGTTPNLMRSVIINCTELVTYDLMKEAFVKNNILADDVPCHLVSALIAGFCATAMSSPVDVVKTRFINSPPGQYKSVPNCAMKVFTNEGPTAFFKGLVPSFLRLGSWNVIMFVCFEQLKRELSKSRQTMDCAT